(19) United States
(12) Patent Application Publication
(10) Pub. No.: US 2020/0040054 A1
(43) Pub. Date: Feb. 6, 2020

(54) ERFE FUSION POLYPEPTIDES COMPOSITIONS AND METHODS OF USE

(71) Applicant: Silarus Therapeutics, Inc., La Jolla, CA (US)

(72) Inventors: Xin DU, La Jolla, CA (US); Vanessa CICCHINI, San Diego, CA (US); Justin CHAPMAN, San Diego, CA (US); Hua WU, San Diego, CA (US); Marc NASOFF, Rancho Santa Fe, CA (US)

(21) Appl. No.: 16/339,295

(22) PCT Filed: Oct. 2, 2017
(86) PCT No.: PCT/US2017/054761

§ 371 Date: Apr. 3, 2019

Related U.S. Application Data
(60) Provisional application No. 62/403,302, filed on Oct. 3, 2016.

(51) Int. Cl. C07K 14/575; C07K 16/18

(52) U.S. Cl. CPC C07K 14/575; A61K 38/00; C07K 16/18

(57) ABSTRACT

Provided herein are ERFE fusion polypeptides, compositions and methods of use for treatment, for example in treatment of iron metabolism disorders.

### RELATED APPLICATIONS

This application claims the benefit of U.S. Provisional Application No. 62/403,302, filed on Oct. 3, 2016, the contents of which are incorporated herein by reference in their entirety.

### SEQUENCE LISTING

The present application is accompanied by a Sequence Listing submitted electronically in ASCII format and which is incorporated by reference in its entirety. Said ASCII copy, created on Sep. 21, 2017, is named 45543_703_601_SL.txt and is 45,472 bytes in size.

### SUMMARY

Described herein are Erythroferrone (ERFE) polypeptides, including ERFE fusion polypeptides, which, for example, in some embodiments, are referred to as ERFE polypeptides, ERFE peptides, ERFE fragments, ERFE polypeptide fragments, ERFE mimetics, fusion proteins, or ERFE truncates. Novel ERFE polypeptides and fusion proteins are provided herein that have ERFE activity including ERFE polypeptides and fusion proteins, that in some embodiments, have ERFE activity including, but not limited to, modulation of hepcidin levels and activity and blood iron levels.

In some aspects, there are provided ERFE fusion polypeptides, comprising (a) an ERFE polypeptide having a sequence at least 85% identical to a polypeptide of SEQ ID NO: 2 or SEQ ID NO: 8, and (b) a heterologous polypeptide. In some embodiments, the ERFE polypeptide comprises at least a fragment of wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of a polypeptide of SEQ ID NO: 2 or SEQ ID NO: 8. In some embodiments, the ERFE polypeptide consists of a fragment of wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of a polypeptide of SEQ ID NO: 2 or SEQ ID NO: 8. In some embodiments, the ERFE polypeptide comprises about 140 to about 320 amino acids at least 85% identical to a polypeptide of SEQ ID NO: 2 or SEQ ID NO: 8. In some embodiments, the ERFE polypeptide has a sequence at least 85% identical to a polypeptide selected from the group consisting of SEQ ID NO: 4, SEQ ID NO: 6, SEQ ID NO: 10, SEQ ID NO: 12, and SEQ ID NO: 14. In some embodiments, the ERFE polypeptide has a sequence at least 90% identical to a polypeptide selected from the group consisting of SEQ ID NO: 4, SEQ ID NO: 6, SEQ ID NO: 10, SEQ ID NO: 12, and SEQ ID NO: 14. In some embodiments, the ERFE polypeptide has a sequence at least 95% identical to a polypeptide selected from the group consisting of SEQ ID NO: 4, SEQ ID NO: 6, SEQ ID NO: 10, SEQ ID NO: 12, and SEQ ID NO: 14. In some embodiments, the ERFE polypeptide has a sequence 99% identical to a polypeptide selected from the group consisting of SEQ ID NO: 4, SEQ ID NO: 6, SEQ ID NO: 10, SEQ ID NO: 12, and SEQ ID NO: 14. In some embodiments, the heterologous polypeptide is selected from the group consisting of calmodulin, polyglutamine, E-tag, FLAG, HA, His, Myc, S-tag, SBP-tag, Softag 1, Softag3, Strep-tag, TC-tag, V5, VSV, Xpress, Isopeptag, SpyTag, SnoopTag, BCCP, GST, GFP, Halo-tag, MBP, Nus-tag, Thioredoxin, albumin, an antibody, Fc domain, and combinations thereof. In some embodiments, the heterologous polypeptide is an Fc domain. In some embodiments, the antibody comprises an anti-albumin antibody. In some embodiments, the antibody targets the fusion polypeptide to a specific cell or tissue. In some embodiments, the heterologous polypeptide is at the N-terminus of the ERFE polypeptide. In some embodiments, the ERFE polypeptide has a sequence at least 85% identical to SEQ ID NO: 4 and the heterologous polypeptide comprises an Fc domain, wherein the heterologous polypeptide is fused to the N-terminus of the ERFE polypeptide. In some embodiments, the ERFE polypeptide has a sequence at least 85% identical to SEQ ID NO: 6 and the heterologous polypeptide comprises an Fc domain, wherein the heterologous polypeptide is fused to the N-terminus of the ERFE polypeptide. In some embodiments, the ERFE polypeptide has a sequence at least 85% identical to SEQ ID NO: 10 and the heterologous polypeptide comprises an Fc domain, wherein the heterologous polypeptide is fused to the N-terminus of the ERFE polypeptide. In some embodiments, the ERFE polypeptide has a sequence at least 85% identical to SEQ ID NO: 12 and the heterologous polypeptide comprises an Fc domain, wherein the heterologous polypeptide is fused to the N-terminus of the ERFE polypeptide. In some embodiments, the heterologous polypeptide is at the C-terminus of the ERFE polypeptide. In some embodiments, the fusion polypeptide modulates ERFE activity. In some embodiments, the polypeptide forms a homo-multimer. In some embodiments, the homo-multimer is a homo-dimer. In some embodiments, there are provided polynucleotides encoding any one of the above fusion polypeptides. In some embodiments, there are provided modified polypeptides comprising any one of the above fusion polypeptides. In some embodiments, the modification is selected from the group consisting of a glycosylation and a phosphorylation. In some embodiments, there are provided compositions comprising any one of the above the fusion polypeptides, the above polynucleotide, or any one of the above modified polypeptides, and an excipient. In some embodiments, the excipient comprises at least one of the group consisting of maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and polyoxyethylene-sorbitan monooleate. In some embodiments, the composition comprises an additional therapeutic agent. In some embodiments, the additional therapeutic agent is iron or erythropoietin. In some embodiments, there are provided any one of the above fusion polypeptides, the above polynucleotide, any one of the above modified polypeptides, or any one of the above compositions for use as a medicament. In some embodiments, there are provided any one of the above fusion polypeptides, the above polynucleotide, any one of the above modified polypeptides, or any one of the above compositions for preparation of a medicament for treatment of a disease or disorder of iron metabolism. In some embodiments, there are provided any one of the above fusion polypeptides, the above polynucleotide, any one of the above modified polypeptides, or any one of the above compositions for use in treatment of a disease or disorder of iron metabolism. In some embodiments, the disease or disorder of iron metabolism is selected from the group consisting of hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia, sideroblastic anemia, porphyria, porphyria cutanea tarda, African iron overload, hyperferritinemia, ceruloplasmin deficiency, atransferrinemia, congenital dyserythropoietic anemia, anemia of chronic disease, anemia of inflammation, anemia of infection, hypochromic microcytic anemia, iron-deficiency anemia, iron-restricted anemia, iron-refractory iron deficiency anemia, anemia of chronic kidney disease, erythropoietin resistance, iron deficiency of obesity, and other anemias. In some embodiments, the disease or disorder of iron metabolism is thalassemia. In some embodiments, the disease or disorder of iron metabolism is thalassemia intermedia. In some embodiments, the disease or disorder of iron metabolism is alpha thalassemia. In some embodiments, the disease or disorder of iron metabolism is beta thalassemia. In certain embodiments, the disease or disorder of iron metabolism is an anemia. In certain embodiments, the diseases or disorders of iron metabolism are iron-restricted anemia, anemia of chronic disease, anemia of inflammation, and anemia of chronic kidney disease. In some embodiments, the disease or disorder of iron metabolism is iron-restricted anemia. In some embodiments, the disease or disorder of iron metabolism is anemia of chronic disease. In some embodiments, the disease or disorder of iron metabolism is anemia of inflammation. In some embodiments, the disease or disorder of iron metabolism is anemia of chronic kidney disease. In some embodiments, the treatment reduces at least one symptom of a disease or disorder of iron metabolism. Symptoms include, but are not limited to, chronic fatigue, joint pain, abdominal pain, liver disease (e.g., cirrhosis, liver cancer), diabetes mellitus, irregular heart rhythm, heart attack or heart failure, skin color changes (e.g., bronze, ashen-gray green), loss of menstrual period, loss of interest in sex, osteoarthritis, osteoporosis, hair loss, enlarged liver or spleen, impotence, infertility, hypogonadism, hypothyroidism, hypopituitarism, depression, adrenal function problems, early onset neurodegenerative disease, elevated blood sugar, elevated liver enzymes, elevated iron (e.g., serum iron, serum ferritin), weakness, pale skin, shortness of breath, dizziness, dietary cravings, tingling or crawling feeling in the legs, tongue swelling or soreness, cold hands and feet, fast or irregular heartbeat, brittle nails, and headache. In some embodiments, the symptom is fatigue. In some embodiments, the symptom is weakness. In some embodiments, the symptom is pale skin. In some embodiments, the symptom is shortness of breath. In some embodiments, the symptom is dizziness.

Also provided herein, in certain aspects, are pharmaceutical compositions, comprising an ERFE polypeptide or ERFE fusion polypeptide and a pharmaceutically acceptable excipient. In some embodiments, the fusion protein comprises (a) an ERFE polypeptide having a sequence at least 85% identical to a fragment of a polypeptide of SEQ ID NO: 2 or SEQ ID NO: 8 and (b) a heterologous polypeptide. In some embodiments, the ERFE polypeptide comprises at least a fragment of wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of a polypeptide of SEQ ID NO: 2 or SEQ ID NO: 8. In some embodiments, the ERFE polypeptide consists of a fragment of wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of a polypeptide of SEQ ID NO: 2 or SEQ ID NO: 8. In some embodiments, the ERFE polypeptide comprises about 140 to about 320 amino acids at least 85% identical to a polypeptide of SEQ ID NO: 2 or SEQ ID NO: 8. In some embodiments, the ERFE polypeptide has a sequence at least 85% identical to a polypeptide selected from the group consisting of SEQ ID NO: 4, SEQ ID NO: 6, SEQ ID NO: 10, SEQ ID NO: 12, and SEQ ID NO: 14. In some embodiments, the ERFE polypeptide has a sequence at least 90% identical to a polypeptide selected from the group consisting of SEQ ID NO: 4, SEQ ID NO: 6, SEQ ID NO: 10, SEQ ID NO: 12, and SEQ ID NO: 14. In some embodiments, the ERFE polypeptide has a sequence at least 95% identical to a polypeptide selected from the group consisting of SEQ ID NO: 4, SEQ ID NO: 6, SEQ ID NO: 10, SEQ ID NO: 12, and SEQ ID NO: 14. In some embodiments, the ERFE polypeptide has a sequence 99% identical to a polypeptide selected from the group consisting of SEQ ID NO: 4, SEQ ID NO: 6, SEQ ID NO: 10, SEQ ID NO: 12, and SEQ ID NO: 14. In some embodiments, the heterologous polypeptide is selected from the group consisting of calmodulin, polyglutamine, E-tag, FLAG, HA, His, Myc, S-tag, SBP-tag, Softag 1, Softag3, Strep-tag, TC-tag, V5, VSV, Xpress, Isopeptag, SpyTag, SnoopTag, BCCP, GST, GFP, Halo-tag, MBP, Nus-tag, Thioredoxin, albumin, an antibody, Fc domain, and combinations thereof. In some embodiments, the heterologous polypeptide is an Fc domain. In some embodiments, the antibody comprises an anti-albumin antibody. In some embodiments, the antibody targets the ERFE polypeptide to a specific cell or tissue. In some embodiments, the heterologous polypeptide is at the N-terminus of the ERFE polypeptide. In some embodiments, the heterologous polypeptide is at the C-terminus of the ERFE polypeptide. In some embodiments, the ERFE fusion polypeptide forms a homo-multimer. In some embodiments, the homo-multimer is a homodimer. In some embodiments, the ERFE fusion polypeptide comprises a modification selected from the group consisting of a glycosylation and a phosphorylation. In some embodiments, the excipient comprises at least one of the group consisting of maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate. In some embodiments, the composition comprises an additional therapeutic agent. In some embodiments, the additional therapeutic agent comprises iron or erythropoietin.

Also provided herein, in certain aspects, are methods of treating a disease or disorder of iron metabolism in an individual in need thereof, comprising administering to the individual a therapeutically-effective amount of an ERFE fusion polypeptide. In some embodiments, the ERFE fusion polypeptide comprises (a) an ERFE polypeptide comprising an ERFE polypeptide having a sequence at least 85% identical to a fragment of a polypeptide of SEQ ID NO: 2 or SEQ ID NO: 8 and (b) a heterologous polypeptide. In some embodiments, the ERFE polypeptide comprises at least a fragment of wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of a polypeptide of SEQ ID NO: 2 or SEQ ID NO: 8. In some embodiments, the ERFE polypeptide consists of a fragment of wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of a polypeptide of SEQ ID NO: 2 or SEQ ID NO: 8. In some embodiments, the ERFE polypeptide comprises about 140 to about 320 amino acids at least 85% identical to a polypeptide of SEQ ID NO: 2 or SEQ ID NO: 8. In some embodiments, the ERFE polypeptide has a sequence at least 85% identical to a polypeptide selected from the group consisting of SEQ ID NO: 4, SEQ ID NO: 6, SEQ ID NO: 10, SEQ ID NO: 12, and SEQ ID NO: 14. In some embodiments, the ERFE polypeptide has a sequence at least 90% identical to a polypeptide selected from the group consisting of SEQ ID NO: 4, SEQ ID NO: 6, SEQ ID NO: 10, SEQ ID NO: 12, and SEQ ID NO: 14. In some embodiments, the ERFE polypeptide has a sequence at least 95% identical to a polypeptide selected from the group consisting of SEQ ID NO: 4, SEQ ID NO: 6, SEQ ID NO: 10, SEQ ID NO: 12, and SEQ ID NO: 14. In some embodiments, the ERFE polypeptide has a sequence 99% identical to a polypeptide selected from the group consisting of SEQ ID NO: 4, SEQ ID NO: 6, SEQ ID NO: 10, SEQ ID NO: 12, and SEQ ID NO: 14. In some embodiments, the heterologous polypeptide is selected from the group consisting of calmodulin, polyglutamine, E-tag, FLAG, HA, His, Myc, S-tag, SBP-tag, Softag 1, Softag3, Strep-tag, TC-tag, V5, VSV, Xpress, Isopeptag, SpyTag, SnoopTag, BCCP, GST, GFP, Halo-tag, MBP, Nus-tag, Thioredoxin, albumin, an antibody, Fc domain, and combinations thereof. In some embodiments, the heterologous polypeptide is an Fc domain. In some embodiments, the antibody comprises an anti-albumin antibody. In some embodiments, the antibody targets an ERFE polypeptide to a specific cell or tissue. In some embodiments, the heterologous polypeptide is at the N-terminus of the ERFE polypeptide. In some embodiments, the heterologous polypeptide is at the C-terminus of the ERFE polypeptide. In some embodiments, the ERFE fusion polypeptide forms a homo-multimer. In some embodiments, the homo-multimer is a homo-dimer. In some embodiments, the ERFE fusion polypeptide comprises a modification selected from the group consisting of a glycosylation and a phosphorylation. In some embodiments, the ERFE fusion polypeptide comprises a composition comprising an ERFE fusion polypeptide and an excipient. In some embodiments, the excipient comprises at least one of the group consisting of maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate. In some embodiments, the method further comprises administering to the individual at least one an additional therapeutic agent. In some embodiments, the additional therapeutic agent is iron or erythropoietin. In some embodiments, the disease or disorder of iron metabolism is selected from the group consisting of hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia, sideroblastic anemia, porphyria, porphyria cutanea tarda, African iron overload, hyperferritinemia, ceruloplasmin deficiency, and atransferrinemia. In some embodiments, the disease or disorder of iron metabolism is selected from the group consisting of congenital dyserythropoietic anemia, anemia of chronic disease, anemia of inflammation, anemia of infection, hypochromic microcytic anemia, iron-deficiency anemia, iron-restricted anemia, iron-refractory iron deficiency anemia, anemia of chronic kidney disease, erythropoietin resistance, iron deficiency of obesity, and other anemias. In some embodiments, the disease or disorder of iron metabolism is thalassemia. In some embodiments, the disease or disorder of iron metabolism is thalassemia intermedia. In some embodiments, the disease or disorder of iron metabolism is alpha thalassemia. In some embodiments, the disease or disorder of iron metabolism is beta thalassemia. In certain embodiments, the disease or disorder of iron metabolism is an anemia. In some embodiments, the method reduces at least one symptom of a disease or disorder of iron metabolism. In some embodiments, the symptom is selected from the group consisting of chronic fatigue, joint pain, abdominal pain, liver disease (e.g., cirrhosis, liver cancer), diabetes mellitus, irregular heart rhythm, heart attack or heart failure, skin color changes (e.g., bronze, ashen-gray green), loss of menstrual period, loss of interest in sex, osteoarthritis, osteoporosis, hair loss, enlarged liver or spleen, impotence, infertility, hypogonadism, hypothyroidism, hypopituitarism, depression, adrenal function problems, early onset neurodegenerative disease, elevated blood sugar, elevated liver enzymes, elevated iron (e.g., serum iron, serum ferritin), weakness, pale skin, shortness of breath, dizziness, dietary cravings, tingling or crawling feeling in the legs, tongue swelling or soreness, cold hands and feet, fast or irregular heartbeat, brittle nails, and headache. In certain embodiments, the diseases or disorders of iron metabolism are iron-restricted anemia, anemia of chronic disease, anemia of inflammation, and anemia of chronic kidney disease. In some embodiments, the disease or disorder of iron metabolism is iron-restricted anemia. In some embodiments, the disease or disorder of iron metabolism is anemia of chronic disease. In some embodiments, the disease or disorder of iron metabolism is anemia of inflammation. In some embodiments, the disease or disorder of iron metabolism is anemia of chronic kidney disease.

Also provided herein, in certain aspects, are kits comprising an ERFE fusion polypeptide and at least one buffer or excipient. In some embodiments, the ERFE fusion polypeptide comprises (a) an ERFE polypeptide having a sequence at least 85% identical to a fragment of a polypeptide of SEQ ID NO: 2 or SEQ ID NO: 8 and (b) a heterologous polypeptide. In some embodiments, the ERFE polypeptide comprises at least a fragment of wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of a polypeptide of SEQ ID NO: 2 or SEQ ID NO: 8. In some embodiments, the ERFE polypeptide consists of a fragment of wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of a polypeptide of SEQ ID NO: 2 or SEQ ID NO: 8. In some embodiments, the ERFE polypeptide comprises about 140 to about 320 amino acids at least 85% identical to a polypeptide of SEQ ID NO: 2 or SEQ ID NO: 8. In some embodiments, the ERFE polypeptide has a sequence at least 85% identical to a polypeptide selected from the group consisting of SEQ ID NO: 4, SEQ ID NO: 6, SEQ ID NO: 10, SEQ ID NO: 12, and SEQ ID NO: 14. In some embodiments, the ERFE polypeptide has a sequence at least 90% identical to a polypeptide selected from the group consisting of SEQ ID NO: 4, SEQ ID NO: 6, SEQ ID NO: 10, SEQ ID NO: 12, and SEQ ID NO: 14. In some embodiments, the ERFE polypeptide has a sequence at least 95% identical to a polypeptide selected from the group consisting of SEQ ID NO: 4, SEQ ID NO: 6, SEQ ID NO: 10, SEQ ID NO: 12, and SEQ ID NO: 14. In some embodiments, the ERFE polypeptide has a sequence 99% identical to a polypeptide selected from the group consisting of SEQ ID NO: 4, SEQ ID NO: 6, SEQ ID NO: 10, SEQ ID NO: 12, and SEQ ID NO: 14. In some embodiments, the heterologous polypeptide is selected from the group consisting of calmodulin, polyglutamine, E-tag, FLAG, HA, His, Myc, S-tag, SBP-tag, Softag 1, Softag3, Strep-tag, TC-tag, V5, VSV, Xpress, Isopeptag, SpyTag, SnoopTag, BCCP, GST, GFP, Halo-tag, MBP, Nus-tag, Thioredoxin, albumin, an antibody, Fc domain, and combinations thereof. In some embodiments, the heterologous polypeptide is an Fc domain. In some embodiments, the antibody comprises an anti-albumin antibody. In some embodiments, the antibody targets the ERFE polypeptide to a specific cell or tissue. In some embodiments, the heterologous polypeptide is at the N-terminus of the ERFE polypeptide. In some embodiments, the heterologous polypeptide is at the C-terminus of the ERFE polypeptide. In some embodiments, the ERFE fusion polypeptide forms a homo-multimer. In some embodiments, the homo-multimer is a homo-dimer. In some embodiments, the ERFE polypeptide comprises a modification selected from the group consisting of a glycosylation and a phosphorylation. In some embodiments, the excipient comprises at least one of the group consisting of maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate. In some embodiments, the kit comprises at least one an additional therapeutic agent. In some embodiments, the additional therapeutic agent comprises iron or erythropoietin. In some embodiments, the kit comprises written instructions for treating a disease or disorder of iron metabolism selected from the group consisting of hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia, sideroblastic anemia, porphyria, porphyria cutanea tarda, African iron overload, hyperferritinemia, ceruloplasmin deficiency, atransferrinemia, congenital dyserythropoietic anemia, anemia of chronic disease, anemia of inflammation, anemia of infection, hypochromic microcytic anemia, iron-deficiency anemia, iron-restricted anemia, iron-refractory iron deficiency anemia, anemia of chronic kidney disease, erythropoietin resistance, iron deficiency of obesity, and other anemias. In some embodiments, the disease or disorder of iron metabolism is thalassemia. In some embodiments, the disease or disorder of iron metabolism is thalassemia intermedia. In some embodiments, the disease or disorder of iron metabolism is alpha thalassemia. In some embodiments, the disease or disorder of iron metabolism is beta thalassemia. In certain embodiments, the disease or disorder of iron metabolism is an anemia. In certain embodiments, the diseases or disorders of iron metabolism are iron-restricted anemia, anemia of chronic disease, anemia of inflammation, and anemia of chronic kidney disease. In some embodiments, the disease or disorder of iron metabolism is iron-restricted anemia. In some embodiments, the disease or disorder of iron metabolism is anemia of chronic disease. In some embodiments, the disease or disorder of iron metabolism is anemia of inflammation. In some embodiments, the disease or disorder of iron metabolism is anemia of chronic kidney disease. In some embodiments, the treatment reduces at least one symptom of a disease or disorder of iron metabolism. Symptoms include, but are not limited to, chronic fatigue, joint pain, abdominal pain, liver disease (e.g., cirrhosis, liver cancer), diabetes mellitus, irregular heart rhythm, heart attack or heart failure, skin color changes (e.g., bronze, ashen-gray green), loss of menstrual period, loss of interest in sex, osteoarthritis, osteoporosis, hair loss, enlarged liver or spleen, impotence, infertility, hypogonadism, hypothyroidism, hypopituitarism, depression, adrenal function problems, early onset neurodegenerative disease, elevated blood sugar, elevated liver enzymes, elevated iron (e.g., serum iron, serum ferritin), weakness, pale skin, shortness of breath, dizziness, dietary cravings, tingling or crawling feeling in the legs, tongue swelling or soreness, cold hands and feet, fast or irregular heartbeat, brittle nails, and headache. In some embodiments, the symptom is fatigue. In some embodiments, the symptom is weakness. In some embodiments, the symptom is pale skin. In some embodiments, the symptom is shortness of breath. In some embodiments, the symptom is dizziness.

### BRIEF DESCRIPTION OF THE DRAWINGS

The novel features are set forth with particularity in the appended claims. A better understanding of the features and advantages herein will be obtained by reference to the following detailed description that sets forth illustrative embodiments, in which the principles herein are utilized, and the accompanying drawings of which:

FIGS. 1A and 1B exemplify structure and deduced functional domains of mERFE Based on Protein Modeling. FIG. 1A exemplifies an amino acid schematic of mERFE and hERFE protein and putative domains. FIG. 1B exemplifies a ribbon diagram of the putative ERFE structure.

FIGS. 2A and 2B exemplify characteristics of Fc_mERFE (24-340). FIG. 2A is a SDS-PAGE gel of Fc_mERFE (24-340). FIG. 2B is an exemplary size exclusion chromatography (SEC) illustrating that a mixture of high molecular weight ERFE polypeptides is present.

FIGS. 3A and 3B exemplify characteristics of Fc_mERFE (24-171). FIG. 3A is a SDS-PAGE gel of Fc_mERFE (24-171). FIG. 3B is an exemplary size exclusion chromatography (SEC) illustrating Fc_mERFE (24-171) predominantly forms dimers under non-denaturing and non-reducing conditions.

FIGS. 4A and 4B exemplify characteristics of Fc_hERFE (43-185) C155/157S. FIG. 4A is a SDS-PAGE gel of Fc-hERFE (43-185) C155/157S. Under reducing conditions Fc_hERFE (43-185) C155/157S ran as monomer, whereas under non-reducing conditions Fc_hERFE (43-185) C155/157S formed only dimers. FIG. 4B is a size exclusion chromatography (SEC) illustrating the predominant presence of dimers.

FIGS. 5A and 5B illustrate functional activity of (FIG. 5A) Fc_mERFE (24-340) and (FIG. 5B) Fc_mERFE (24-171) in Hep3B cellular HAMP suppression assay. Error bar represents individual assay carried out in duplicate.

FIGS. 6A-C illustrate functional activity of (FIG. 6A) Fc_hERFE (43-354), (FIG. 6B) Fc_hERFE (43-185) and (FIG. 6C) Fc_hERFE (43-185) C155/157S in Hep3B cellular HAMP suppression assay. Error bar represents individual assay carried out in duplicate.

FIGS. 7A-C illustrate mERFE in vivo activity. Liver Hamp transcript and serum hepcidin levels were reduced at 6 hours post administration of 10 mg/kg of (FIG. 7A) Fc_mERFE (24-340), or (FIG. 7B) Fc_mERFE (24-171); FIG. 7C shows serum iron levels elevation due to the decrease in hepcidin levels at 6 hours post injection. N=5 for Fc_mERFE (24-340) group, n=8 for Fc_mERFE (24-171) group. Results are expressed at Mean±SEM.

FIGS. 8A and 8B illustrate in vivo dose-response of Fc_mERFE (24-171). Liver Hamp transcript (FIG. 8A) and serum hepcidin levels (FIG. 8B) were analyzed for mice at 6 hours after receiving 1 mg/kg, 5 mg/kg, and 10 mg/kg of Fc_mERFE (24-171) by IP injection. 8 mice per group, and response of each individual mouse is shown in the scatter plot.

FIG. 9 illustrates functional activity of Flag-His mERFE (24-340) in the presence of IL-6 in Hep3B cellular HAMP suppression assay. Error bar represents individual assay carried out in duplicate.

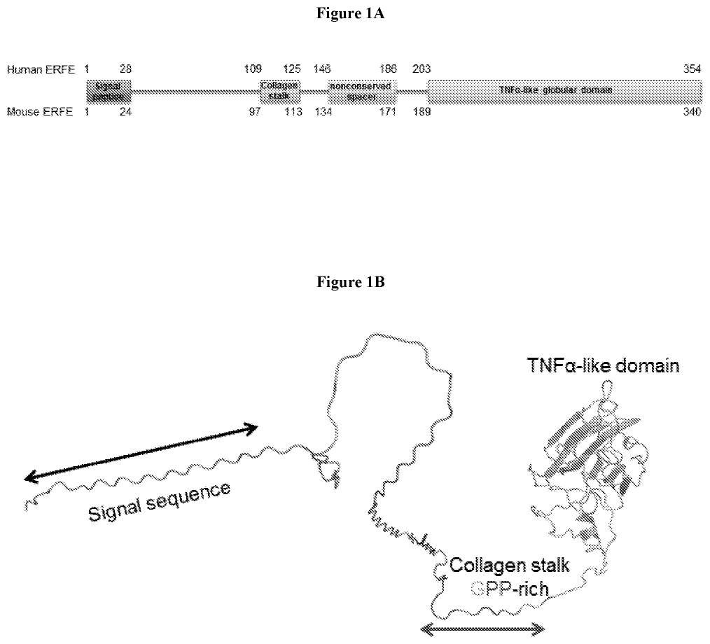

### DETAILED DESCRIPTION

Disclosed herein, in some embodiments, are ERFE fusion polypeptides comprising (a) an ERFE polypeptide having a sequence at least 85% identical to a fragment of a polypeptide of SEQ ID NO: 2 or SEQ ID NO: 8, and (b) a heterologous polypeptide. Further disclosed herein, in some embodiments, are compositions and pharmaceutical compositions, comprising an ERFE polypeptide or ERFE fusion polypeptide and an excipient. Additionally disclosed herein, in some embodiments, are methods of treating a disease or disorder of iron metabolism in an individual in need thereof, comprising administering to the individual a therapeutically-effective amount of an ERFE fusion polypeptide. Also disclosed herein, in some embodiments are ERFE fusion polypeptides for use in preparation of a medicament. Further disclosed herein are ERFE fusion polypeptides for use in preparation of a medicament for treatment of a disease or disorder of iron metabolism. Additionally disclosed herein are ERFE fusion polypeptides for use in treatment of a disease or disorder of iron metabolism. Further disclosed herein, in some embodiments, are kits comprising an ERFE fusion polypeptide and at least one buffer or excipient.

### Definitions

As used herein, erythroferrone (including ERFE and Erfe) and its analogs and fragments are collectively referred to herein as, e.g., “ERFE polypeptides”. As used herein, “ERFE activity” refers to the ability of a substance to decrease, e.g., by at least about 10%, at least about 20%, at least about 50%, at least about 70%, at least about 90%, or more, hepatic hepcidin mRNA or serum hepcidin levels as compared to a control.

As used herein, the terms “protein,” “polypeptide,” and “peptide” are used interchangeably to refer to two or more amino acids linked together.

In some embodiments, the ERFE polypeptides herein are substantially purified. As used herein, a “substantially purified” compound or an “isolated” compound, used interchangeably herein, refers to a compound that is removed from its natural environment and/or is at least about 60% free, about 75% free, about 90% free, or about 95-100% free from other macromolecular components or compounds with which the compound is associated with in nature or from its synthesis.

As used herein, the term “modulate,” and grammatical variations thereof, when used in reference to an ERFE activity or function, means that the ERFE activity or function is detectably affected, altered or changed, e.g., as compared to an untreated state, e.g., by at least about 10%, at least about 20%, at least about 50%, at least about 70%, at least about 90%, or more compared to the untreated state. Thus, an ERFE polypeptide that modulates an ERFE activity or function is a polypeptide that detectably affects, alters or changes one or more ERFE activities or functions, which, in some embodiments, includes, for example, binding of ERFE to an ERFE receptor, ERFE mediated signaling or an ERFE-mediated or ERFE-modulatable cell response, or another ERFE activity or function as set forth herein or otherwise known or knowable.

As used herein, the term “subsequence” or “fragment” means a portion of the full length molecule. A subsequence of an ERFE polypeptide encoding an ERFE polypeptide has at least one fewer amino acids than a full length ERFE (e.g., one or more internal or terminal amino acid deletions from either amino or carboxy-termini). A subsequence of ERFE polypeptide has at least one fewer amino acid than a full length ERFE polypeptide. A nucleic acid subsequence has at least one less nucleotide than a full length comparison nucleic acid sequence. Subsequences therefore in some embodiments are any length from at least about 10, at least about 20, at least about 30, at least about 40, at least about 50, at least about 60, at least about 70, at least about 80, at least about 90, at least about 100, at least about 110, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 310, at least about 320, at least about 330, at least about 340, or more amino acids amino acids up to the full length native ERFE. Exemplary human ERFE polypeptide fragments include, but are not limited to, 43-53, 43-73, 43-93, 43-110, 43-120, 43-130, 43-140, 43-150, 43-155, 43-160, 43-165, 43-170, 43-175, 43-180, 43-181, 43-182, 43-183, 43-184, 43-185, 43-186, 43-187, 43-188, 43-189, 43-190, 43-195, 43-200, 43-205, 43-210, 43-215, 43-220, 43-230, 43-240, 43-250, 43-260, 43-270, 43-280, 43-290, 43-300, 43-310, 43-320, 43-330, 43-340, 43-350, 43-351, 43-352, 43-353, 43-43-354, 40-354, 41-354, 42-354, 43-354, 28-354, 29-354, 30-354, 31-354, 32-354, 33-354, 34-354, 35-354, 36-354, 37-354, 38-354, 39-354, 40-354, 41-354, 42-354, 43-354, 44-354, 45-354, 46-354, 47-354, 48-354, 49-354, 50-354, 55-354, 60-354, 65-354, 70-354, 75-354, 80-354, 85-354, 90-354, 100-354, 110-354, 120-354, 130-354, 140-354, 150-354, 160-354, 165-354, 170-354, 175-354, 180-354, 190-354, 200-354, 210-354, 220-354, 230-354, 240-354, 250-354, 260-354, 270-354, 280-354, 290-354, 300-354, 310-354, 320-354, 325-354, 330-354, 335-354, 340-354, 341-354, 342-354, 343-354, 344-354, 28-185, 29-185, 30-185, 31-185, 32-185, 33-185, 34-185, 35-185, 36-185, 37-185, 38-185, 39-185, 40-185, 41-185, 42-185, 43-185, 44-185, 45-185, 46-185, 47-185, 48-185, 49-185, 50-185, 55-185, 60-185, 65-185, 70-185, 75-185, 80-185, 85-185, 90-185, 100-185, 110-185, 120-185, 130-185, 140-185, 150-185, 160-185, 165-185, 170-185, 171-185, 172-185, 173-185, 174-185, and 175-185.

Pharmaceutical formulations include “pharmaceutically acceptable” and “physiologically acceptable” carriers, diluents or excipients. The terms “pharmaceutically acceptable” and “physiologically acceptable” include solvents (aqueous or non-aqueous), solutions, emulsions, dispersion media, coatings, isotonic and absorption promoting or delaying agents, compatible with pharmaceutical administration to a mammal, for example a human. In some embodiments, such formulations are contained in a liquid, e.g., emulsion, suspension, syrup or elixir; or solid form, i.e., tablet (e.g., coated or uncoated, immediate, delayed, continuous, or pulsatile release), capsule (e.g., hard or soft, immediate, delayed, continuous, or pulsatile release), powder, granule, crystal, or microbead. In some embodiments, supplementary compounds (e.g., preservatives, antibacterial, antiviral and antifungal agents) are also incorporated into the formulations.

As used herein, all numerical values or numerical ranges include whole integers within or encompassing such ranges and fractions of the values or the integers within or encompassing ranges unless the context clearly indicates otherwise. Thus, for example, reference to a range of 90-100%, includes 91%, 92%, 93%, 94%, 95%, 95%, 97%, etc., as well as 91.1%, 91.2%, 91.3%, 91.4%, 91.5%, etc., 92.1%, 92.2%, 92.3%, 92.4%, 92.5%, etc., and so forth. In another example, reference to a range of 1-5,000 fold includes 1, 2, 3, 4, 5, 6, 7, 8, 9, 10, 11, 12, 13, 14, 15, 16, 17, 18, 19, 20, fold, etc., as well as 1.1, 1.2, 1.3, 1.4, 1.5, fold, etc., 2.1, 2.2, 2.3, 2.4, 2.5, fold, etc., and so forth.

As used herein, singular forms “a,” “and,” and “the” include plural referents unless the context clearly indicates otherwise. Thus, for example, reference to “a polypeptide” includes a plurality of polypeptides and reference to “a treatment or therapy” in some embodiments includes multiple, sequential or simultaneous treatments or therapies, and so forth.

### Erythroferrone

Erythroferrone is the first identified “hormone” that mediates between red blood cell production and the absorption and distribution of iron in individuals (e.g., mammals, e.g., humans). Erythroferrone is made in the bone marrow of an individual and its production is greatly increased when the production of red blood cells is stimulated, e.g., after bleeding or during recovery from anemia. Erythroferrone regulates the supply of iron to meet the needs of red blood cell production in the marrow. Specifically, erythroferrone is found to act on the liver to suppress the production of the principal iron-regulatory protein, hepcidin. Thus, in some embodiments, overproduction of erythroferrone causes iron overload in diseases such as β-thalassemia and antagonizing erythroferrone is used for the treatment of β-thalassemia.

Erythroferrone was identified in the search for an erythroid factor that suppresses hepcidin expression. Hepcidin, a 25 amino acid peptide hormone synthesized by the liver, is the central regulator of iron homeostasis. Hepcidin acts by binding to the sole iron exporter ferroportin leading to its ubiquitination, internalization and degradation in lysosomes. When ferroportin disappears from the cell membranes, dietary iron absorption is inhibited and recycled iron is sequestered in macrophages, decreasing iron availability for erythropoiesis. In contrast, low hepcidin allows ferroportin to remain active on cells that export iron to plasma, making more iron available for hemoglobin synthesis. Iron, inflammation, or ER stress stimulates hepcidin production, whereas hypoxia, iron deficiency and increased erythropoietic activity repress it.

Hepcidin is suppressed after hemorrhage or erythropoietin (EPO) administration. Hepcidin is decreased in anemia caused by bleeding, hemolysis, iron deficiency, or ineffective erythropoiesis. The suppressive effect of erythropoiesis on hepcidin is particularly prominent in diseases with ineffective erythropoiesis where erythrocyte precursors massively expand but mostly undergo apoptosis at the erythroblast stage rather than mature into erythrocytes.

ERFE is also referred to as Complement C1q tumor necrosis factor-related protein 15, Myonectin, FAM132B, C1QTNF15 and CTRP15. ERFE includes mammalian (e.g., primate, murine, human) forms of ERFE. ERFE polypeptides include polypeptides that modulate ERFE activities. One non-limiting example of a full length human ERFE is a sequence set forth as:

(SEQ ID NO: 8)
MAPARRPAGARLLLVYAGLLAAAAAGLGSPEPGAPSRSRARREPPPGNEL
PRGPGESRAGPAARPPEPTAERAHSVDPRDAWMLFVRQSDKGVNGKKRSR
GKAKKLKFGLPGPPGPPGPQGPPGPIIPPEALLKEFQLLLKGAVRQRERA
EPEPCTCGPAGPVAASLAPVSATAGEDDDDVVGDVLALLAAPLAPGPRAP
RVEAAFLCRLRRDALVERRALHELGVYYLPDAEGAFRRGPGLNLTSGQYR
APVAGFYALAATLHVALGEPPRRGPPRPRDHLRLLICIQSRCQRNASLEA
IMGLESSSELFTISVNGVLYLQMGQWTSVFLDNASGCSLTVRSGSHFSAV
LLGV.

### ERFE Polypeptide Fragments

Provided herein, are ERFE polypeptides consisting of a fragment of an ERFE polypeptide having a sequence set forth in SEQ ID NO: 4, SEQ ID NO: 6, SEQ ID NO: 10, SEQ ID NO: 12, or SEQ ID NO: 14. In some embodiments, an ERFE polypeptide fragment has a sequence at least 50% identical to a fragment of a polypeptide of SEQ ID NO: 4, SEQ ID NO: 6, SEQ ID NO: 10, SEQ ID NO: 12, or SEQ ID NO: 14. In some embodiments, an ERFE polypeptide fragment has a sequence at least 30%, 40%, 50%, 60%, 70%, 75%, 80%, 85%, 90%, 91%, 92%, 93%, 94%, 95%, 96%, 97%, 98%, 99%, or 100% identical to a fragment of a polypeptide of SEQ ID NO: 4, SEQ ID NO: 6, SEQ ID NO: 10, SEQ ID NO: 12, or SEQ ID NO: 14. In some embodiments, an ERFE polypeptide fragment has a sequence at least 85% identical to a polypeptide selected from the group consisting of SEQ ID NO: 4, SEQ ID NO: 6, SEQ ID NO: 10, SEQ ID NO: 12, and SEQ ID NO: 14. In some embodiments, an ERFE polypeptide fragment has a sequence at least 90% identical to a polypeptide selected from the group consisting of SEQ ID NO: 4, SEQ ID NO: 6, SEQ ID NO: 10, SEQ ID NO: 12, and SEQ ID NO: 14. In some embodiments, an ERFE polypeptide fragment has a sequence at least 95% identical to a polypeptide selected from the group consisting of SEQ ID NO: 4, SEQ ID NO: 6, SEQ ID NO: 10, SEQ ID NO: 12, and SEQ ID NO: 14. In some embodiments, an ERFE polypeptide fragment has a sequence at least 99% identical to a polypeptide selected from the group consisting of SEQ ID NO: 4, SEQ ID NO: 6, SEQ ID NO: 10, SEQ ID NO: 12, and SEQ ID NO: 14. In some embodiments, an ERFE polypeptide fragment has a sequence at least 100% identical to a polypeptide selected from the group consisting of SEQ ID NO: 4, SEQ ID NO: 6, SEQ ID NO: 10, SEQ ID NO: 12, and SEQ ID NO: 14.

In some embodiments, ERFE polypeptides herein comprise fragment of a wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of a polypeptide of SEQ ID NO: 2 or SEQ ID NO: 8. In some embodiments, ERFE polypeptides herein comprise a fragment of a wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of a polypeptide of SEQ ID NO: 2 or SEQ ID NO: 8. In some embodiments, an ERFE polypeptide comprises a fragment of a wildtype ERFE having about 140 to about 320 amino acids of a polypeptide of SEQ ID NO: 2 or SEQ ID NO: 8. In some embodiments, an ERFE polypeptide comprises a fragment of a wildtype ERFE having about 140 to about 320 amino acids of a polypeptide of SEQ ID NO: 2 or SEQ ID NO: 8.

Due to variation between structurally and functionally related proteins, such as ERFE polypeptide fragments, the amount of sequence identity required to retain a function or activity depends upon the protein, the region and the function or activity of that region. Although, in some embodiments, there is as little as 30% amino acid sequence identity for ERFE polypeptide fragments, to retain a given activity or function, typically there is more, e.g., 50%, 60%, 75%, 85%, 90%, 95%, 96%, 97%, 98%, identity to a wildtype reference sequence. The extent of identity between two sequences, in some embodiments, is ascertained using a computer program and mathematical algorithm known in the art. Such algorithms that calculate percent sequence identity (homology) generally account for sequence gaps and mismatches over the comparison region. For example, a BLAST (e.g., BLAST 2.0) search algorithm (see, e.g., Altschul et al., J. Mol. Biol. 215:403 (1990), publicly available through NCBI) has exemplary search parameters as follows: Mismatch-2; gap open 5; gap extension 2. For polypeptide sequence comparisons, a BLASTP algorithm is typically used in combination with a scoring matrix, such as PAM100, PAM 250, BLOSUM 62 or BLOSUM 50. FASTA (e.g., FASTA2 and FASTA3) and SSEARCH sequence comparison programs are also used to quantitate the extent of identity (Pearson et al., Proc. Natl. Acad. Sci. USA 85:2444 (1988); Pearson, Methods Mol Biol. 132:185 (2000); and Smith et al., J. Mol. Biol. 147:195 (1981)).

Also disclosed herein, in certain embodiments, are modified ERFE polypeptide fragments. In some embodiments, a modified ERFE polypeptide fragment comprises a glycosylated ERFE polypeptide fragment. In some embodiments, a modified ERFE polypeptide fragment comprises a phosphorylated ERFE polypeptide fragment. In some embodiments, the modification is selected from the group consisting of: myristoylation, palmitoylation, isoprenylation, glypiation, lipolation, acylation, akylation, amidation, phosphorylation, glycation, biotinylation, pegylation, sumoylation, ubiquitination, neddylation, or pupylation. Modifications also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond.

Also disclosed herein, in certain embodiments, are polynucleotides encoding an ERFE polypeptide fragment disclosed herein. In some embodiments, the polynucleotide encodes at least a fragment of a polypeptide of SEQ ID NO: 4, SEQ ID NO: 6, SEQ ID NO: 10, SEQ ID NO: 12, or SEQ ID NO: 14. In some embodiments, the polynucleotide encodes a polypeptide at least 30%, 40%, 50%, 60%, 70%, 75%, 80%, 85%, 90%, 91%, 92%, 93%, 94%, 95%, 96%, 97%, 98%, 99%, or 100% identical to a fragment of a polypeptide of SEQ ID NO: 4, SEQ ID NO: 6, SEQ ID NO: 10, SEQ ID NO: 12, or SEQ ID NO: 14. In some embodiments, the polynucleotide encodes a polypeptide comprising a fragment of an ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of a polypeptide of SEQ ID NO: 4, SEQ ID NO: 6, SEQ ID NO: 10, SEQ ID NO: 12, or SEQ ID NO: 14. In some embodiments, the polynucleotide comprises a promoter sequence. In some embodiments, the polynucleotide comprises a heterologous promoter. In some embodiments, the polynucleotide comprises an inducible promoter sequence. In some embodiments, the polynucleotide comprises a plasmid. In some embodiments, the polynucleotide comprises a viral vector such as a retroviral vector, a lentiviral vector, an adenoviral vector, or an adeno-associated viral vector. In some embodiments, a cell comprises a polynucleotide (e.g., vector) disclosed herein.

Also disclosed herein, in certain embodiments, are cells expressing ERFE polypeptide fragments disclosed herein. In some embodiments, the cell is a mammalian cell. In some embodiments, the cell is an insect cell. In some embodiments, the cell is a yeast cell. In some embodiments, the cell is a bacterial cell. Examples of cells for expressing an ERFE polypeptide disclosed herein include, but are not limited to, a CHO cell, a ExpiCHO-S cell, a CHO DG44 cell, a CHO-K1 cell, a myeloma cell, a hybridoma cell, a NS0 cell, a GS-NSO cell, aHEK293 cell, a HEK293T cell, aHEK293E cell, a HEK293-6E cell, a HEK293F cell, and a per.C6 cell. In some embodiments, the cell is a CHO cell. In some embodiments, the cell is a myeloma cell. In some embodiments, the cell is selected from the group consisting of an E. coli cell, a P. mirabilis cell, a P. putidas cell, a B. brevis cell, a B. megaterium cell, a B. subtilis cell, a L. paracasei cell, a S. lividans cell, a Y. lipolytica cell, a K. lactis cell, a P. pastoris cell, a S. cerevisiae cell, a A. niger var. awamori cell, a A. oryzae cell, a L. tarentolae cell, a T. ni larvae cell, a S. frugiperda cell, a Drosophila S2 cell, a S. frugiperda SF9 cell, a T. ni cell, and a SfSWT-1 mimic cell.

### ERFE Fusion Polypeptides

Disclosed herein, in certain embodiments, are ERFE fusion polypeptides comprising: (a) an ERFE polypeptide and (b) at least one heterologous protein or fragment thereof.

ERFE fusion polypeptides comprising an ERFE polypeptide comprise at least a fragment of an ERFE polypeptide having a sequence set forth in SEQ ID NO: 2 or SEQ ID NO: 8. In some embodiments, a fusion protein comprises an ERFE polypeptide has a sequence at least 50% identical to a fragment of a polypeptide of SEQ ID NO: 2 or SEQ ID NO: 8. In some embodiments, a fusion protein comprises an ERFE polypeptide has a sequence at least 30%, 40%, 50%, 60%, 70%, 75%, 80%, 85%, 90%, 91%, 92%, 93%, 94%, 95%, 96%, 97%, 98%, 99%, or 100% identical to a fragment of a polypeptide of SEQ ID NO: 2 or SEQ ID NO: 8. In some embodiments, a fusion protein comprises an ERFE polypeptide has a sequence at least 60%, 70%, 75%, 80%, 85%, 90%, 91%, 92%, 93%, 94%, 95%, 96%, 97%, 98%, 99%, or 100% identical to a polypeptide selected from the group consisting of SEQ ID NO: 4, SEQ ID NO: 6, SEQ ID NO: 10, SEQ ID NO: 12, and SEQ ID NO: 14. In some embodiments, a fusion protein comprises an ERFE polypeptide has a sequence at least 85% identical to a fragment of a polypeptide of SEQ ID NO: 2 or SEQ ID NO: 8. In some embodiments, a fusion protein comprises an ERFE polypeptide has a sequence at least 85% identical to a polypeptide selected from the group consisting of SEQ ID NO: 4, SEQ ID NO: 6, SEQ ID NO: 10, SEQ ID NO: 12, and SEQ ID NO: 14. In some embodiments, a fusion protein comprises an ERFE polypeptide has a sequence at least 90% identical to a polypeptide selected from the group consisting of SEQ ID NO: 4, SEQ ID NO: 6, SEQ ID NO: 10, SEQ ID NO: 12, and SEQ ID NO: 14. In some embodiments, a fusion protein comprises an ERFE polypeptide has a sequence at least 95% identical to a polypeptide selected from the group consisting of SEQ ID NO: 4, SEQ ID NO: 6, SEQ ID NO: 10, SEQ ID NO: 12, and SEQ ID NO: 14. In some embodiments, a fusion protein comprises an ERFE polypeptide has a sequence at least 99% identical to a polypeptide selected from the group consisting of SEQ ID NO: 4, SEQ ID NO: 6, SEQ ID NO: 10, SEQ ID NO: 12, and SEQ ID NO: 14. In some embodiments, a fusion protein comprises an ERFE polypeptide has a sequence 100% identical to a polypeptide selected from the group consisting of SEQ ID NO: 4, SEQ ID NO: 6, SEQ ID NO: 10, SEQ ID NO: 12, and SEQ ID NO: 14.

In some embodiments, a fusion protein comprises a ERFE polypeptide comprising a fragment of a wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of a polypeptide of SEQ ID NO: 2 or SEQ ID NO: 8. In some embodiments, a fusion protein comprises a ERFE polypeptide consisting of a fragment of a wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of a polypeptide of SEQ ID NO: 2 or SEQ ID NO: 8. In some embodiments, a fusion protein comprises a ERFE polypeptide comprising about 140 to about 320 amino acids of a polypeptide of SEQ ID NO: 2 or SEQ ID NO: 8. In some embodiments, a fusion protein comprises a ERFE polypeptide consisting of about 140 to about 320 amino acids of a polypeptide of SEQ ID NO: 2 or SEQ ID NO: 8.

Also disclosed herein, in certain embodiments, are modified ERFE fusion polypeptides. In some embodiments, a modified ERFE fusion polypeptide comprises a glycosylated ERFE fusion polypeptide. In some embodiments, a modified ERFE polypeptide fragment comprises a phosphorylated ERFE fusion polypeptide. In some embodiments, the modification is selected from the group consisting of: myristoylation, palmitoylation, isoprenylation, glypiation, lipolation, acylation, akylation, amidation, phosphorylation, glycation, biotinylation, pegylation, sumoylation, ubiquitination, neddylation, or pupylation. Modifications also include one or more D-amino acids substituted for L-amino acids (and mixtures thereof), structural and functional analogues, for example, peptidomimetics having synthetic or non-natural amino acids or amino acid analogues and derivatized forms. Modifications include cyclic structures such as an end-to-end amide bond between the amino and carboxy-terminus of the molecule or intra- or inter-molecular disulfide bond.

ERFE fusion polypeptides herein comprise heterologous proteins. Any suitable heterologous protein is contemplated for use in the fusion proteins disclosed herein. In some embodiments, the heterologous protein is selected from the group consisting of calmodulin, polyglutamine, E-tag, FLAG, HA, His, Myc, S-tag, SBP-tag, Softag 1, Softag3, Strep-tag, TC-tag, V5, VSV, Xpress, Isopeptag, SpyTag, SnoopTag, BCCP, GST, GFP, Halo-tag, MBP, Nus-tag, Thioredoxin, albumin, an antibody, Fc domain, and combinations thereof. In some embodiments, the heterologous protein comprises an Fc domain. In some embodiments, the heterologous protein comprises a His tag. In some embodiments, the heterologous protein comprises a FLAG. In some embodiments, the heterologous protein comprises a His tag and a FLAG. In some embodiments, the heterologous protein comprises an antibody. In some embodiments, the heterologous protein comprises an antibody that targets the fusion protein to a particular cell or tissue. In some embodiments, the heterologous protein comprises an anti-albumin antibody.

Heterologous proteins herein have sequences available to those of skill in the art. Exemplary sequences of heterologous proteins are provided in Table 1 below.

TABLE 1
Heterologous Protein Sequences

| | | SEQ |
|---|---|---|
| Heterologous | | ID |
| Protein | Sequence | NO: |
| Calmodulin | MADQLTEEQIAEFKEAFSLFDKDGDGTITTKELGTVMRSLGQN | 15 |
| | PTEAELQDMINEVDADGNGTIDFPEFLTMMARKMKDTDSEEEI | |
| | REAFRVFDKDGNGYISAAELRHVMTNLGEKLTDEEVDEMIREA | |
| | DIDGDGQVNYEEFVQMMTAK | |
| Polyglutamine | EEEEEE | 16 |
| E-tag | GAPVPYPDPLEPR | 17 |
| FLAG | DYKDDDDK | 18 |
| HA | YPYDVPDYA | 19 |
| His | HHHHHH | 20 |
| Myc | EQKLISEEDL | 21 |
| NE-tag | TKENPRSNQEESYDDNES | 22 |
| S-tag | KETAAAKFERQHMDS | 23 |
| SBP-tag | MDEKTTGWRGGHVVEGLAGELEQLRARLEHHPQGQREP | 24 |
| Softag 1 | SLAELLNAGLGGS | 25 |
| Softag 3 | TQDPSRVG | 26 |
| Streptag | WSHPQFEK | 27 |
| TC tag | CCPGCC | 28 |
| V5 | GKPIPNPLLGLDST | 29 |
| VSV | YTDIEMNRLGK | 30 |
| Xpress | DLYDDDDK | 31 |
| Isopeptag | TDKDMTITFTNKKDAE | 32 |
| SpyTag | AHIVMVDAYKPTK | 33 |
| SnoopTag | KLGDIEFIKVNK | 34 |
| BCCP | AAAEISGHIVRSPMVGTFYRTPSPDAKAFIEVGQKVNVGDTLC | 35 |
| | IVEAMKMMNQIEADKSGTVKAILVESGQPVEFDEPLVVIE | |
| GST | MSPILGYWKIKGLVQPTRLLLEYLEEKYEEHLYERDEGDKWRN | 36 |
| | KKFELGLEFPNLPYYIDGDVKLTQSMAIIRYIADKHNMLGGCP | |
| | KERAEISMLEGAVLDIRYGVSRIAYSKDFETLKVDFLSKLPEM | |
| | LKMFEDRLCHKTYLNGDHVTHPDFMLYDALDVVLYMDPMCLDA | |
| | FPKLVCFKKRIEAIPQIDKYLKSSKYIAWPLQGWQATFGGGDH | |
| | PPKSDLVPRGSPGIHRD | |
| GFP | MVSKGEELFTGVVPILVELDGDVNGHKFSVSGEGEGDATYGKL | 37 |
| | TLKFICTTGKLPVPWPTLVTTLTYGVQCFSRYPDHMKQHDFFK | |
| | SAMPEGYVQERTIFFKDDGNYKTRAEVKFEGDTLVNRIELKGI | |
| | DFKEDGNILGHKLEYNYNSHNVYIMADKQKNGIKVNFKIRHNI | |
| | EDGSVQLADHYQQNTPIGDGPVLLPDNHYLSTQSALSKDPNEK | |
| | RDHMVLLEFVTAAGITLGMDELYK | |
| MBP | MKIEEGKLVIWINGDKGYNGLAEVGKKFEKDTGIKVTVEHPDK | 38 |
| | LEEKFPQVAATGDGPDIIFWAHDRFGGYAQSGLLAEITPDKAF | |
| | QDKLYPFTWDAVRYNGKLIAYPIAVEALSLIYNKDLLPNPPKT | |
| | WEEIPALDKELKAKGKSALMFNLQEPYFTWPLIAADGGYAFKY | |
| | ENGKYDIKDVGVDNAGAKAGLTFLVDLIKNKHMNADTDYSIAE | |
| | AAFNKGETAMTINGPWAWSNIDTSKVNYGVTVLPTFKGQPSKP | |
| | FVGVLSAGINAASPNKELAKEFLENYLLTDEGLEAVNKDKPLG | |
| | AVALKSYEEELAKDPRIAATMENAQKGEIMPNIPQMSAFWYAV | |
| | RTAVINAASGRQTVDEALKDAQTNSSSNNNNNNNNNNLGIEGR | |
| Albumin | MKWVTFISLLFLFSSAYSRGVFRRDAHKSEVAHRFKDLGEENF | 39 |
| | KALVLIAFAQYLQQCPFEDHVKLVNEVTEFAKTCVADESAENC | |
| | DKSLHTLFGDKLCTVATLRETYGEMADCCAKQEPERNECFLQH | |
| | KDDNPNLPRLVRPEVDVMCTAFHDNEETFLKKYLYEIARRHPY | |
| | FYAPELLFFAKRYKAAFTECCQAADKAACLLPKLDELRDEGKA | |
| | SSAKQGLKCASLQKFGERAFKAWAVARLSQRFPKAEFAEVSKL | |
| | VTDLTKVHTECCHGDLLECADDRADLAKYICENQDSISSKLKE | |
| | CCEKPLLEKSHCIAEVENDEMPADLPSLAADFVGSKDVCKNYA | |
| | EAKDVFLGMFLYEYARRHPDYSVVLLLRLAKTYETTLEKCCAA | |
| | ADPHECYAKVFDEFKPLVEEPQNLIKQNCELFEQLGEYKFQNA | |
| | LLVRYTKKVPQVSTPTLVEVSRNLGKVGSKCCKHPEAKRMPCA | |
| | EDCLSVFLNQLCVLHEKTPVSDRVTKCCTESLVNGRPCFSALE | |
| | VDETYVPKEFNAETFTFHADICTLSEKERQIKKQTALVELVKH | |
| | KPKATKEQLKAVMDDFAAFVEKCCKADDKETCFAEEGKKLVAA | |
| | SQAALGL | |
| Fc domain | EPKSCDKTHTCPPCPAPELLGGPSVFLFPPKPKDTLMISRTPE | 40 |
| | VTCVVVDVSHEDPEVKFNWYVDGVEVHNAKTKPREEQYNSTYR | |
| | VVSVLTVLHQDWLNGKEYKCKVSNKALPAPIEKTISKAKGQPR | |
| | EPQVYTLPPSREEMTKNQVSLTCLVKGFYPSDIAVEWESNGQP | |
| | ENNYKTTPPVLDSDGSFFLYSKLTVDKSRWQQGNVFSCSVMHE | |
| | ALHNHYTQKSLSLSPGK | |

ERFE fusion polypeptides herein, in some embodiments, comprise (a) an ERFE polypeptide selected from the group consisting of SEQ ID NO: 4, SEQ ID NO: 6, SEQ ID NO: 10, SEQ ID NO: 12, and SEQ ID NO: 14, and (b) an Fc domain. In some embodiments, the Fc domain is fused to the N-terminus of the ERFE polypeptide. In some embodiments, the ERFE fusion polypeptide comprises an ERFE polypeptide having a sequence of SEQ ID NO: 4 and an N-terminal Fc domain. In some embodiments, the ERFE fusion polypeptide comprises an ERFE polypeptide having a sequence of SEQ ID NO: 6 and an N-terminal Fc domain. In some embodiments, the ERFE fusion polypeptide comprises an ERFE polypeptide having a sequence of SEQ ID NO: 10 and an N-terminal Fc domain. In some embodiments, the ERFE fusion polypeptide comprises an ERFE polypeptide having a sequence of SEQ ID NO: 12 and an N-terminal Fc domain.

Also disclosed herein, in certain embodiments, are cells expressing ERFE fusion polypeptides disclosed herein. In some embodiments, the cell is a mammalian cell. In some embodiments, the cell is an insect cell. In some embodiments, the cell is a yeast cell. In some embodiments, the cell is a bacterial cell. Examples of cells for expressing an ERFE fusion polypeptide disclosed herein include, but are not limited to, a CHO cell, a ExpiCHO-S cell, a CHO DG44 cell, a CHO-K1 cell, a myeloma cell, a hybridoma cell, a NSO cell, a GS-NSO cell, aHEK293 cell, a HEK293T cell, aHEK293E cell, a HEK293-6E cell, a HEK293F cell, and a per.C6 cell. In some embodiments, the cell is a CHO cell. In some embodiments, the cell is a myeloma cell. In some embodiments, the cell is selected from the group consisting of an E. coli cell, a P. mirabilis cell, a P. putidas cell, a B. brevis cell, a B. megaterium cell, a B. subtilis cell, a L. paracasei cell, a S. lividans cell, a Y. lipolytica cell, a K. lactis cell, a P. pastoris cell, a S. cerevisiae cell, a A. niger var. awamori cell, a A. oryzae cell, a L. tarentolae cell, a T. ni larvae cell, a S. frugiperda cell, a Drosophila S2 cell, a S. frugiperda SF9 cell, a T. ni cell, and a SfSWT-1 mimic cell.

### ERFE Polypeptide Compositions and Formulations

Also disclosed herein, in certain embodiments, are compositions comprising an ERFE polypeptide disclosed herein or an EFRE fusion protein disclosed herein, and an excipient.

In some embodiments, excipients for use with the compositions disclosed herein include maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate. In some embodiments, the composition further comprises a carrier.

In some embodiments, the compositions further comprise an additional therapeutic agent. In some embodiments, the additional therapeutic agent treats a symptom of a disease or disorder of iron metabolism. In some embodiments, the additional therapeutic agent increases the efficacy of the modulator of ERFE activity. In some embodiments, the composition further comprises iron. In some embodiments, the composition further comprises erythropoietin.

Pharmaceutical formulations, in some embodiments, are made to be compatible with a particular local, regional or systemic administration or delivery route. Thus, pharmaceutical formulations include carriers, diluents, or excipients suitable for administration by particular routes. Specific non-limiting examples of routes of administration for compositions herein are parenteral, e.g., intravenous, intra-arterial, intradermal, intramuscular, subcutaneous, intra-pleural, transdermal (topical), transmucosal, intra-cranial, intra-spinal, intra-ocular, rectal, oral (alimentary), mucosal administration, and any other formulation suitable for the treatment method or administration protocol.

In some embodiments, solutions or suspensions used for parenteral application include: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfate; chelating agents such as ethylenediaminetetraacetic acid; buffers such as acetates, citrates or phosphates; and agents for the adjustment of tonicity such as sodium chloride or dextrose. In some embodiments, pH is adjusted with acids or bases, such as hydrochloric acid or sodium hydroxide.

Pharmaceutical formulations for injection include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.), or phosphate buffered saline (PBS). In some embodiments, the carrier is a solvent or dispersion medium containing, for example, water, ethanol, polyol (for example, glycerol, propylene glycol, and liquid polyetheylene glycol, and the like), or suitable mixtures thereof. Fluidity is maintained, in some embodiments, for example, by the use of a coating such as lecithin, by the maintenance of the required particle size in the case of dispersion, and by the use of surfactants. Antibacterial and antifungal agents include, for example, parabens, chlorobutanol, phenol, ascorbic acid, and thimerosal. Isotonic agents, for example, sugars; polyalcohols such as mannitol or sorbitol; or sodium chloride, in some embodiments, are included in the composition. In some cases, also included is an agent which delays absorption, in some embodiments, for example, aluminum monostearate or gelatin prolongs absorption of injectable compositions.

In some embodiments, sterile injectable formulations are prepared by incorporating the active composition in the required amount in an appropriate solvent with one or a combination of above ingredients. Generally, dispersions are prepared by incorporating the active composition into a sterile vehicle containing a basic dispersion medium and any other ingredient. In the case of sterile powders for the preparation of sterile injectable solutions, methods of preparation include, for example, vacuum drying and freeze-drying which yields a powder of the active ingredient plus any additional desired ingredient from a previously prepared solution thereof.

For transmucosal or transdermal administration, penetrants appropriate to the barrier to be permeated are used in the formulation. Such penetrants are known in the art, and include, for example, for transmucosal administration, detergents, bile salts, and fusidic acid derivatives. In some embodiments, transmucosal administration is accomplished through the use of nasal sprays, inhalation devices (e.g., aspirators) or suppositories. For transdermal administration, the active compounds are formulated into ointments, salves, gels, creams or patches.

In some embodiments, the pharmaceutical formulations are prepared with carriers that protect against rapid elimination from the body, such as a controlled release formulation or a time delay material such as glyceryl monostearate or glyceryl stearate. The formulations, in some embodiments, are also delivered using articles of manufacture such as implants and microencapsulated delivery systems to achieve local, regional or systemic delivery or controlled or sustained release.

### Methods and Uses in Treatment of Diseases of Iron Metabolism

Also disclosed herein, in certain embodiments, are methods of treating diseases or disorders of iron metabolism in an individual in need thereof comprising administering to the individual a composition comprising an ERFE polypeptide or ERFE fusion polypeptide. In some embodiments, the method reduces at least one symptom of a disease or disorder of iron metabolism. In some embodiments, there are provided compositions comprising an ERFE polypeptide or ERFE fusion polypeptide for use as a medicament. In some embodiments, there are provided compositions comprising an ERFE polypeptide or ERFE fusion polypeptide for preparation of a medicament for treatment of a disease or disorder of iron metabolism. In some embodiments, there are provided any one of the above fusion polypeptides, the above polynucleotide, any one of the above modified polypeptides, or any one of the above compositions for use in treatment of a disease or disorder of iron metabolism.

Also provided herein, in certain aspects, are ERFE polypeptides and ERFE fusion polypeptides that modulate ERFE activity. In some embodiments, ERFE polypeptides and ERFE fusion polypeptides herein modulate an ERFE function or activity in vivo or in vitro (e.g. in an individual). In some embodiments, an ERFE polypeptide increases ERFE activity. ERFE polypeptides herein that increase ERFE activity comprise at least a fragment of ERFE having ERFE activity. Fragments of ERFE having ERFE activity can be determined using conventional methods and methods described herein. In some embodiments, increasing ERFE activity comprises decreasing hepcidin activity. In some embodiments, increasing ERFE activity comprises decreasing hepcidin mRNA expression. In some embodiments, increasing ERFE activity comprises increasing blood iron levels in an individual, e.g., in an individual in need thereof.

In some embodiments, an ERFE polypeptide or fusion protein decreases ERFE activity. In some embodiments, an ERFE polypeptide or fusion protein that decreases ERFE activity is acting as an inhibitor of ERFE. In some embodiments, an ERFE polypeptide or fusion protein that decreases ERFE activity is a competitive antagonist. In some embodiments, decreasing ERFE activity comprises increasing hepcidin activity. In some embodiments, decreasing ERFE activity comprises increasing hepcidin mRNA expression. In some embodiments, decreasing ERFE activity comprises decreasing serum iron levels in an individual.

In some embodiments, the disease or disorder of iron metabolism is hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia, sideroblastic anemia, porphyria, porphyria cutanea tarda, African iron overload, hyperferritinemia, ceruloplasmin deficiency, atransferrinemia, congenital dyserythropoietic anemia, anemia of chronic disease, anemia of inflammation, anemia of infection, hypochromic microcytic anemia, iron-deficiency anemia, iron-restricted anemia, iron-refractory iron deficiency anemia, anemia of chronic kidney disease, erythropoietin resistance, iron deficiency of obesity, or another anemia. In some embodiments, the disease or disorder of iron metabolism is thalassemia. In some embodiments, the disease or disorder of iron metabolism is thalassemia intermedia. In some embodiments, the disease or disorder of iron metabolism is alpha thalassemia. In some embodiments, the disease or disorder of iron metabolism is beta thalassemia. In certain embodiments, the disease or disorder of iron metabolism is an anemia. In certain embodiments, the diseases or disorders of iron metabolism are iron-restricted anemia, anemia of chronic disease, anemia of inflammation, or anemia of chronic kidney disease. In some embodiments, the disease or disorder of iron metabolism is iron-restricted anemia. In some embodiments, the disease or disorder of iron metabolism is anemia of chronic disease. In some embodiments, the disease or disorder of iron metabolism is anemia of inflammation. In some embodiments, the disease or disorder of iron metabolism is anemia of chronic kidney disease.

In some embodiments, the disease or disorder of iron metabolism is iron-restricted anemia, as in the cases of anemia of inflammation and anemia of chronic disease.

In some embodiments, the method reduces at least one symptom of a disease or disorder of iron metabolism. Symptoms include, but are not limited to, chronic fatigue, joint pain, abdominal pain, liver disease (e.g., cirrhosis, liver cancer), diabetes mellitus, irregular heart rhythm, heart attack or heart failure, skin color changes (e.g., bronze, ashen-gray green), loss of menstrual period, loss of interest in sex, osteoarthritis, osteoporosis, hair loss, enlarged liver or spleen, impotence, infertility, hypogonadism, hypothyroidism, hypopituitarism, depression, adrenal function problems, early onset neurodegenerative disease, elevated blood sugar, elevated liver enzymes, elevated iron (e.g., serum iron, serum ferritin), weakness, pale skin, shortness of breath, dizziness, dietary cravings, tingling or crawling feeling in the legs, tongue swelling or soreness, cold hands and feet, fast or irregular heartbeat, brittle nails, and headache. In some embodiments, the symptom is fatigue. In some embodiments, the symptom is weakness. In some embodiments, the symptom is pale skin. In some embodiments, the symptom is shortness of breath. In some embodiments, the symptom is dizziness.

Any suitable route of administration is contemplated for use with the methods disclosed herein. In some embodiments, the composition is administered by intravenous administration. In some embodiments, the composition is administered locally. In some embodiments, the composition is administered systemically (e.g., intravenously, intramuscularly, subcutaneously, intradermally, orally, intranasally, sublingually). In some embodiments, the composition is formulated as a salve, lotion or emulsion. In some embodiments, the composition is formulated as a solution. In some embodiments, the composition is formulated for topical, oral, buccal, or nasal administration.

Compositions are administered singly, or over a time course, such as daily, multiple times weekly, weekly, biweekly, monthly or less frequently. Compositions are administered alone or in concert with additional measures that are in some cases related to treatment of the disease or a symptom thereof, such as dietary supplement or adjustment, exercise or other treatment. Administration occurs during or between meals, and is independent of, or alternately dependent upon, daily administration timing, such as morning administration, evening administration, or multiple administrations relative to sleep, meals or exercise.

In some embodiments, the individual is monitored prior to administration of the composition. Symptoms are identified and their severity is assessed. A composition as described herein is administered alone or in combination with additional treatments, singly or multiply over time as discussed herein or known to one of skill in the art. In some embodiments, the individual is monitored such that the efficacy of the treatment regimen is determined. In some embodiments, a treatment regimen is modified in response to preliminary treatment outcomes, such that treatment dose or frequency or dose and frequency is altered so as to attain a desired level of subject response in light of symptom alleviation, side effect reduction, or a combination of symptom alleviation and side effect reduction.

Therapeutically effective amounts or dosages are contemplated to include dosages of 0.01 mg to 20 mg, for example, 0.01 mg, 0.02 mg, 0.03 mg, 0.04 mg, 0.05 mg, 0.06 mg, 0.07 mg, 0.08 mg, 0.09 mg, 0.1 mg, 0.2 mg, 0.3 mg, 0.4 mg, 0.5 mg, 0.6 mg, 0.7 mg, 0.8 mg, 0.9 mg, 1.0 mg, 1.1 mg, 1.2 mg, 1.3 mg, 1.4 mg, 1.5 mg, 1.6 mg, 1.7 mg, 1.8 mg, 1.9 mg, 2 mg, 2.1 mg, 2.2 mg, 2.3 mg, 2.4 mg, 2.5 mg, 2.6 mg, 2.7 mg, 2.8 mg, 2.9 mg, 3 mg, 3.1 mg, 3.2 mg, 3.3 mg, 3.4 mg, 3.5 mg, 3.6 mg, 3.7 mg, 3.8 mg, 3.9 mg, 4 mg, 4.1 mg, 4.2 mg, 4.3 mg, 4.4 mg, 4.5 mg, 4.6 mg, 4.7 mg, 4.8 mg, 4.9 mg, 5 mg, 5.1 mg, 5.2 mg, 5.3 mg, 5.4 mg, 5.5 mg, 5.6 mg, 5.7 mg, 5.8 mg, 5.9 mg, 6 mg, 6.1 mg, 6.2 mg, 6.3 mg, 6.4 mg, 6.5 mg, 6.6 mg, 6.7 mg, 6.8 mg, 6.9 mg, 7 mg, 7.1 mg, 7.2 mg, 7.3 mg, 7.4 mg, 7.5 mg, 7.6 mg, 7.7 mg, 7.8 mg, 7.9 mg, 8 mg, 8.1 mg, 8.2 mg, 8.3 mg, 8.4 mg, 8.5 mg, 8.6 mg, 8.7 mg, 8.8 mg, 8.9 mg, 9 mg, 9.1 mg, 9.2 mg, 9.3 mg, 9.4 mg, 9.5 mg, 9.6 mg, 9.7 mg, 9.8 mg, 9.9 mg, 10 mg, 10.1 mg, 10.2 mg, 10.3 mg, 10.4 mg, 10.5 mg, 10.6 mg, 10.7 mg, 10.8 mg, 10.9 mg, 11 mg, 11.1 mg, 11.2 mg, 11.3 mg, 11.4 mg, 11.5 mg, 11.6 mg, 11.7 mg, 11.8 mg, 11.9 mg, 12 mg, 12.1 mg, 12.2 mg, 12.3 mg, 12.4 mg, 12.5 mg, 12.6 mg, 12.7 mg, 12.8 mg, 12.9 mg, 13 mg, 13.1 mg, 13.2 mg, 13.3 mg, 13.4 mg, 13.5 mg, 13.6 mg, 13.7 mg, 13.8 mg, 13.9 mg, 14 mg, 14.1 mg, 14.2 mg, 14.3 mg, 14.4 mg, 14.5 mg, 14.6 mg, 14.7 mg, 14.8 mg, 14.9 mg, 15 mg, 15.1 mg, 15.2 mg, 15.3 mg, 15.4 mg, 15.5 mg, 15.6 mg, 15.7 mg, 15.8 mg, 15.9 mg, 16 mg, 16.1 mg, 16.2 mg, 16.3 mg, 16.4 mg, 16.5 mg, 16.6 mg, 16.7 mg, 16.8 mg, 16.9 mg, 17 mg, 17.1 mg, 17.2 mg, 17.3 mg, 17.4 mg, 17.5 mg, 17.6 mg, 17.7 mg, 17.8 mg, 17.9 mg, 18 mg, 18.1 mg, 18.2 mg, 18.3 mg, 18.4 mg, 18.5 mg, 18.6 mg, 18.7 mg, 18.8 mg, 18.9 mg, 19 mg, 19.1 mg, 19.2 mg, 19.3 mg, 19.4 mg, 19.5 mg, 19.6 mg, 19.7 mg, 19.8 mg, 19.9 mg, or 20 mg. Therapeutically effective amounts or dosages, in some cases, are contemplated to include dosages of 0.1 mg to 2.0 mg.

### ERFE Fusion Polypeptide Kits

Also disclosed herein, in certain embodiments, are kits comprising an ERFE polypeptide disclosed herein or an ERFE fusion polypeptide disclosed herein, and at least one buffer or excipient. Alternatively or in combination, kits herein comprise nucleic acids encoding an ERFE polypeptide or an ERFE fusion polypeptide, and pharmaceutical formulations thereof. Kits herein are packaged into suitable packaging material, optionally in combination with instructions for using the kit components, e.g., instructions for performing a method herein. In some embodiments, a kit comprises an ERFE polypeptide or ERFE fusion polypeptide and instructions for treating an individual in need of treatment (e.g., in some embodiments, an individual having a disease, disorder, pathology, or condition amenable or that responds to treatment or therapy) with the ERFE polypeptide or fusion protein. For example, in some embodiments, the kits include drugs and compositions for diagnosing, treating, or monitoring disorders of iron metabolism. In some embodiments, a kit comprising a composition comprising an ERFE polypeptide or fusion protein also comprises reagents for determining serum iron levels in a subject and instructions for carrying out an assay for determining serum iron levels. In some embodiments, a kit comprises reagents for carrying out an assay for determining serum iron levels or for diagnosing a disease or disorder of iron metabolism, exemplary reagents comprising igepal, ferene S, hydroxylamine chloride, and iron (III) chloride; and an ERFE polypeptide or fusion protein for treating the disease or disorder of iron metabolism.

In some embodiments, a kit herein comprises a buffering agent, a preservative, or a stabilizing agent. In some embodiments, the kit includes control components for assaying for activity, e.g., a control sample or a standard. In some embodiments, each component of the kit is enclosed within an individual container or in a mixture and all of the various containers in certain embodiments are within single or multiple packages.

In some embodiments, instructions additionally include indications of a satisfactory clinical endpoint or any adverse symptoms or complications that occur in some embodiments. In some embodiments, instructions further include storage information, expiration date, or any information required by regulatory agencies such as the Food and Drug Administration for use in a human individual.

A number of embodiments have been described herein. Nevertheless, it will be understood that, in some embodiments, various modifications are made without departing from the spirit and scope herein. Accordingly, the following examples are intended to illustrate but not limit the scope described in the claims.

### EXAMPLES

### Example 1: ERFE Fusion Polypeptides

Recombinant mouse and human ERFE proteins, designated mERFE and hERFE (respectively) are made by cloning ERFE mRNA into a mammalian expression vector followed by transient expression, for example in FreeStyle 293-F cells (Invitrogen). The functional activity of recombinant ERFE molecules is evaluated by a cellular assay based on ERFE-induced suppression of Hepcidin mRNA (HAMP) adapted from the published methodology. In addition to full-length ERFE protein, a series of ERFE truncates have been created in order to identify the necessary peptide sequences that are responsible for the hepcidin-suppressing activity. It has been found that ERFE functional activity resides in the N-terminal region of the protein, and the following active ERFE variants are identified: mERFE (24-340) (SEQ ID NO: 4), mERFE (24-171) (SEQ ID NO: 6), hERFE (43-354) (SEQ ID NO: 10), hERFE (43-185) (SEQ ID NO: 12), and hERFE (43-185) C155/157S (SEQ ID NO: 14) (the numbering refers to the amino acid coordinates of ERFE protein sequence). hERFE (43-185) C155/1575 is a variant version of hERFE (43-185) in which cysteines at position 155 and 157 were changed to serines.

Active ERFE variants are expressed as fusion proteins with N-terminus attached to a tag, such as a (Flag)3-(His)6 tag ((His)6 tag” disclosed as SEQ ID NO: 20), or fused to the Fc portion of human IgG1 to achieve extended serum half-life. Examples below are from analyses on mERFE and hERFE Fc-fusion proteins and mERFE Flag-His-fusion proteins.

TABLE 2
Active mouse and human ERFE molecules

| SEQ | | |
|---|---|---|
| ID | | |
| NO: | Description | Sequence |
| 1 | mERFE (1- | ATGGCCTCGACCCGCCGCCCCGTCGGAGCTCGCACGCTGCTCGCC |
| | 340) cDNA | TGCGCCAGCCTACTCGCCGCCATGGGCCTCGGTGTCCCTGAGTCC |
| | | GCGGAGCCCGTGGGGACTCATGCACGCCCGCAGCCGCCCGGGGCC |
| | | GAGCTGCCCGCCCCGCCAGCCAACAGCCCGCCGGAACCCACCATT |
| | | GCGCATGCACACAGTGTGGATCCCCGGGATGCTTGGATGCTGTTC |
| | | GTCAAGCAGAGTGACAAGGGGATCAACAGTAAGAGGAGGAGCAAA |
| | | GCCAGGAGGCTGAAGCTTGGCCTGCCAGGACCCCCAGGGCCACCA |
| | | GGTCCTCAGGGCCCCCCAGGCCCCTTTATCCCATCTGAGGTTCTG |
| | | CTGAAGGAGTTCCAGCTGTTGCTGAAAGGCGCAGTACGGCAGCGA |
| | | GAGAGCCATCTGGAGCACTGCACCAGGGATCTCACTACACCAGCC |
| | | TCGGGTAGCCCTTCCCGTGTCCCAGCCGCCCAGGAGCTTGATAGC |
| | | CAGGACCCAGGGGCATTGTTAGCTCTGCTGGCTGCGACCTTGGCC |
| | | CAGGGCCCGCGGGCACCACGTGTGGAGGCCGCATTCCACTGTCGC |
| | | TTGCGCCGGGATGTGCAGGTGGATCGGCGTGCGTTGCACGAGCTT |
| | | GGGATCTACTACCTGCCCGAAGTTGAGGGAGCCTTCCACCGGGGC |
| | | CCAGGCTTGAATCTGACCAGCGGCCAGTACACCGCACCTGTGGCT |
| | | GGCTTCTATGCGCTTGCTGCCACTCTGCACGTGGCACTCACCGAG |
| | | CAGCCAAGAAAGGGACCAACACGACCCCGGGATCGTCTGCGCCTG |
| | | CTGATCTGCATCCAGTCTCTCTGCCAGCACAATGCCTCCCTGGAG |
| | | ACTGTGATGGGGCTGGAGAACAGCAGCGAGCTCTTCACCATCTCA |
| | | GTAAATGGTGTCCTCTATCTACAGGCAGGACACTACACTTCTGTC |
| | | TTCTTGGACAATGCCAGCGGCTCCTCCCTCACGGTACGCAGTGGC |
| | | TCTCACTTCAGTGCTATCCTCCTGGGCCTGTGA |
| 2 | mERFE (1- | MASTRRPVGARTLLACASLLAAMGLGVPESAEPVGTHARPQPPGA |
| | 340) amino | ELPAPPANSPPEPTIAHAHSVDPRDAWMLFVKQSDKGINSKRRSK |
| | acid | ARRLKLGLPGPPGPPGPQGPPGPFIPSEVLLKEFQLLLKGAVRQR |
| | | ESHLEHCTRDLTTPASGSPSRVPAAQELDSQDPGALLALLAATLA |
| | | QGPRAPRVEAAFHCRLRRDVQVDRRALHELGIYYLPEVEGAFHRG |
| | | PGLNLTSGQYTAPVAGFYALAATLHVALTEQPRKGPTRPRDRLRL |
| | | LICIQSLCQHNASLETVMGLENSSELFTISVNGVLYLQAGHYTSV |
| | | FLDNASGSSLTVRSGSHFSAILLGL |
| 3 | mERFE (24- | ATGGGCCTCGGTGTCCCTGAGTCCGCGGAGCCCGTGGGGACTCAT |
| | 340) cDNA | GCACGCCCGCAGCCGCCCGGGGCCGAGCTGCCCGCCCCGCCAGCC |
| | | AACAGCCCGCCGGAACCCACCATTGCGCATGCACACAGTGTGGAC |
| | | CCCCGGGATGCTTGGATGCTGTTCGTCAAGCAGAGTGACAAGGGG |
| | | ATCAACAGTAAGAGGAGGAGCAAAGCCAGGAGGCTGAAGCTTGGC |
| | | CTGCCAGGACCCCCAGGGCCACCAGGTCCTCAGGGCCCCCCAGGC |
| | | CCCTTTATCCCATCTGAGGTTCTGCTGAAGGAGTTCCAGCTGTTG |
| | | CTGAAAGGCGCAGTACGGCAGCGAGAGAGCCATCTGGAGCACTGC |
| | | ACCAGGGATCTCACTACACCAGCCTCGGGTAGCCCTTCCCGTGTC |
| | | CCAGCCGCCCAGGAGCTTGATAGCCAGGACCCAGGGGCATTGTTA |
| | | GCTCTGCTGGCTGCGACCTTGGCCCAGGGCCCGCGGGCACCACGT |
| | | GTGGAGGCCGCATTCCACTGTCGCTTGCGCCGGGATGTGCAGGTG |
| | | GATCGGCGTGCGTTGCACGAGCTTGGGATCTACTACCTGCCCGAA |
| | | GTTGAGGGAGCCTTCCACCGGGGCCCAGGCTTGAATCTGACCAGC |
| | | GGCCAGTACACCGCACCTGTGGCTGGCTTCTATGCGCTTGCTGCC |
| | | ACTCTGCACGTGGCACTCACCGAGCAGCCAAGAAAGGGACCAACA |
| | | CGACCCCGGGATCGTCTGCGCCTGCTGATCTGCATCCAGTCTCTC |
| | | TGCCAGCACAATGCCTCCCTGGAGACTGTGATGGGGCTGGAGAAC |
| | | AGCAGCGAGCTCTTCACCATCTCAGTAAATGGTGTCCTCTATCTA |
| | | CAGGCAGGACACTACACTTCTGTCTTCTTGGACAATGCCAGCGGC |
| | | TCCTCCCTCACGGTACGCAGTGGCTCTCACTTCAGTGCTATCCTC |
| | | CTGGGCCTGTGA |
| 4 | mERFE (24- | MGLGVPESAEPVGTHARPQPPGAELPAPPANSPPEPTIAHAHSVD |
| | 340) amino | PRDAWMLFVKQSDKGINSKRRSKARRLKLGLPGPPGPPGPQGPPG |
| | acid | PFIPSEVLLKEFQLLLKGAVRQRESHLEHCTRDLTTPASGSPSRV |
| | | PAAQELDSQDPGALLALLAATLAQGPRAPRVEAAFHCRLRRDVQV |
| | | DRRALHELGIYYLPEVEGAFHRGPGLNLTSGQYTAPVAGFYALAA |
| | | TLHVALTEQPRKGPTRPRDRLRLLICIQSLCQHNASLETVMGLEN |
| | | SSELFTISVNGVLYLQAGHYTSVFLDNASGSSLTVRSGSHFSAIL |
| | | LGL |
| 5 | mERFE (24- | ATGGGCCTCGGTGTCCCTGAGTCCGCGGAGCCCGTGGGGACTCAT |
| | 171) cDNA | GCACGCCCGCAGCCGCCCGGGGCCGAGCTGCCCGCCCCGCCAGCC |
| | | AACAGCCCGCCGGAACCCACCATTGCGCATGCACACAGTGTGGAC |
| | | CCCCGGGATGCTTGGATGCTGTTCGTCAAGCAGAGTGACAAGGGG |
| | | ATCAACAGTAAGAGGAGGAGCAAAGCCAGGAGGCTGAAGCTTGGC |
| | | CTGCCAGGACCCCCAGGGCCACCAGGTCCTCAGGGCCCCCCAGGC |
| | | CCCTTTATCCCATCTGAGGTTCTGCTGAAGGAGTTCCAGCTGTTG |
| | | CTGAAAGGCGCAGTACGGCAGCGAGAGAGCCATCTGGAGCACTGC |
| | | ACCAGGGATCTCACTACACCAGCCTCGGGTAGCCCTTCCCGTGTC |
| | | CCAGCCGCCCAGGAGCTTGATAGCCAGGACCCAGGGGCATTGTGA |
| 6 | mERFE (24- | MGLGVPESAEPVGTHARPQPPGAELPAPPANSPPEPTIAHAHSVD |
| | 171) amino | PRDAWMLFVKQSDKGINSKRRSKARRLKLGLPGPPGPPGPQGPPG |
| | acid | PFIPSEVLLKEFQLLLKGAVRQRESHLEHCTRDLTTPASGSPSRV |
| | | PAAQELDSQDPGAL |
| 7 | hERFE (1- | ATGGCCCCGGCCCGCCGCCCCGCCGGAGCCCGCCTGCTGCTCGTC |
| | 354) cDNA | TACGCGGGCCTGCTGGCCGCCGCCGCCGCGGGCCTGGGGTCCCCG |
| | | GAGCCTGGGGCGCCCTCGAGGAGCCGCGCCCGCAGGGAGCCGCCG |
| | | CCCGGGAACGAGCTGCCCCGGGGCCCCGGGGAGAGCCGCGCGGGG |
| | | CCGGCCGCTCGTCCGCCGGAGCCCACCGCTGAGCGTGCACACAGC |
| | | GTCGACCCCCGGGACGCCTGGATGCTCTTCGTCAGGCAGAGTGAC |
| | | AAGGGTGTCAATGGCAAGAAGAGGAGCAGGGGCAAGGCCAAGAAG |
| | | CTGAAGTTCGGCTTGCCAGGGCCCCCTGGGCCTCCCGGTCCCCAG |
| | | GGCCCCCCAGGCCCCATCATCCCACCCGAGGCGCTGCTGAAGGAG |
| | | TTCCAGCTGCTGCTGAAAGGTGCGGTGCGGCAGCGGGAGCGCGCG |
| | | GAGCCCGAACCCTGTACGTGTGGCCCCGCCGGGCCGGTCGCTGCG |
| | | AGCCTCGCCCCGGTCTCGGCCACCGCCGGGGAGGACGACGACGAC |
| | | GTGGTGGGGGACGTGCTGGCACTGCTGGCCGCGCCCCTGGCCCCG |
| | | GGGCCGCGGGCGCCGCGCGTGGAGGCCGCTTTCCTCTGCCGCCTG |
| | | CGCCGGGACGCGTTGGTGGAGCGGCGCGCGCTGCACGAGCTTGGC |
| | | GTCTACTACCTGCCCGACGCCGAGGGTGCCTTCCGCCGCGGCCCG |
| | | GGCCTGAACTTGACCAGCGGCCAGTACAGGGCGCCCGTGGCTGGC |
| | | TTCTACGCTCTCGCCGCCACGCTGCACGTGGCGCTCGGGGAGCCG |
| | | CCGAGGAGGGGGCCGCCGCGCCCCCGGGACCACCTGCGCCTGCTC |
| | | ATCTGCATCCAGTCCCGGTGCCAGCGCAACGCCTCCCTGGAGGCC |
| | | ATCATGGGCCTGGAGAGCAGCAGTGAGCTCTTCACCATCTCTGTG |
| | | AATGGCGTCCTGTACCTGCAGATGGGGCAGTGGACCTCCGTGTTC |
| | | TTGGACAACGCCAGCGGCTGCTCCCTCACAGTGCGCAGTGGCTCC |
| | | CACTTCAGTGCTGTCCTCCTGGGCGTGTGA |
| 8 | hERFE (1- | MAPARRPAGARLLLVYAGLLAAAAAGLGSPEPGAPSRSRARREPP |
| | 354) amino | PGNELPRGPGESRAGPAARPPEPTAERAHSVDPRDAWMLFVRQSD |
| | acid | KGVNGKKRSRGKAKKLKFGLPGPPGPPGPQGPPGPIIPPEALLKE |
| | | FQLLLKGAVRQRERAEPEPCTCGPAGPVAASLAPVSATAGEDDDD |
| | | VVGDVLALLAAPLAPGPRAPRVEAAFLCRLRRDALVERRALHELG |
| | | VYYLPDAEGAFRRGPGLNLTSGQYRAPVAGFYALAATLHVALGEP |
| | | PRRGPPRPRDHLRLLICIQSRCQRNASLEAIMGLESSSELFTISV |
| | | NGVLYLQMGQWTSVFLDNASGCSLTVRSGSHFSAVLLGV |
| 9 | hERFE (43- | GAGCCGCCGCCCGGGAACGAGCTGCCCCGGGGCCCCGGGGAGAGC |
| | 354) cDNA | CGCGCGGGGCCGGCCGCTCGTCCGCCGGAGCCCACCGCTGAGCGT |
| | | GCACACAGCGTCGACCCCCGGGACGCCTGGATGCTCTTCGTCAGG |
| | | CAGAGTGACAAGGGTGTCAATGGCAAGAAGAGGAGCAGGGGCAAG |
| | | GCCAAGAAGCTGAAGTTCGGCTTGCCAGGGCCCCCTGGGCCTCCC |
| | | GGTCCCCAGGGCCCCCCAGGCCCCATCATCCCACCCGAGGCGCTG |
| | | CTGAAGGAGTTCCAGCTGCTGCTGAAAGGTGCGGTGCGGCAGCGG |
| | | GAGCGCGCGGAGCCCGAACCCTGTACGTGTGGCCCCGCCGGGCCG |
| | | GTCGCTGCGAGCCTCGCCCCGGTCTCGGCCACCGCCGGGGAGGAC |
| | | GACGACGACGTGGTGGGGGACGTGCTGGCACTGCTGGCCGCGCCC |
| | | CTGGCCCCGGGGCCGCGGGCGCCGCGCGTGGAGGCCGCTTTCCTC |
| | | TGCCGCCTGCGCCGGGACGCGTTGGTGGAGCGGCGCGCGCTGCAC |
| | | GAGCTTGGCGTCTACTACCTGCCCGACGCCGAGGGTGCCTTCCGC |
| | | CGCGGCCCGGGCCTGAACTTGACCAGCGGCCAGTACAGGGCGCCC |
| | | GTGGCTGGCTTCTACGCTCTCGCCGCCACGCTGCACGTGGCGCTC |
| | | GGGGAGCCGCCGAGGAGGGGGCCGCCGCGCCCCCGGGACCACCTG |
| | | CGCCTGCTCATCTGCATCCAGTCCCGGTGCCAGCGCAACGCCTCC |
| | | CTGGAGGCCATCATGGGCCTGGAGAGCAGCAGTGAGCTCTTCACC |
| | | ATCTCTGTGAATGGCGTCCTGTACCTGCAGATGGGGCAGTGGACC |
| | | TCCGTGTTCTTGGACAACGCCAGCGGCTGCTCCCTCACAGTGCGC |
| | | AGTGGCTCCCACTTCAGTGCTGTCCTCCTGGGCGTGTGA |
| 10 | hERFE (43- | EPPPGNELPRGPGESRAGPAARPPEPTAERAHSVDPRDAWMLFVR |
| | 354) amino | QSDKGVNGKKRSRGKAKKLKFGLPGPPGPPGPQGPPGPIIPPEAL |
| | acid | LKEFQLLLKGAVRQRERAEPEPCTCGPAGPVAASLAPVSATAGED |
| | | DDDVVGDVLALLAAPLAPGPRAPRVEAAFLCRLRRDALVERRALH |
| | | ELGVYYLPDAEGAFRRGPGLNLTSGQYRAPVAGFYALAATLHVAL |
| | | GEPPRRGPPRPRDHLRLLICIQSRCQRNASLEAIMGLESSSELFT |
| | | ISVNGVLYLQMGQWTSVFLDNASGCSLTVRSGSHFSAVLLGV |
| 11 | hERFE (43- | GAGCCGCCGCCCGGGAACGAGCTGCCCCGGGGCCCCGGGGAGAGC |
| | 185) cDNA | CGCGCGGGGCCGGCCGCTCGTCCGCCGGAGCCCACCGCTGAGCGT |
| | | GCACACAGCGTCGACCCCCGGGACGCCTGGATGCTCTTCGTCAGG |
| | | CAGAGTGACAAGGGTGTCAATGGCAAGAAGAGGAGCAGGGGCAAG |
| | | GCCAAGAAGCTGAAGTTCGGCTTGCCAGGGCCCCCTGGGCCTCCC |
| | | GGTCCCCAGGGCCCCCCAGGCCCCATCATCCCACCCGAGGCGCTG |
| | | CTGAAGGAGTTCCAGCTGCTGCTGAAAGGTGCGGTGCGGCAGCGG |
| | | GAGCGCGCGGAGCCCGAACCCTGTACGTGTGGCCCCGCCGGGCCG |
| | | GTCGCTGCGAGCCTCGCCCCGGTCTCGGCCACCGCCGGGGAGGAC |
| | | GACGACGACGTGGTGGGGGACGTGTGA |
| 12 | hERFE (43- | EPPPGNELPRGPGESRAGPAARPPEPTAERAHSVDPRDAWMLFVR |
| | 185) amino | QSDKGVNGKKRSRGKAKKLKFGLPGPPGPPGPQGPPGPIIPPEAL |
| | acid | LKEFQLLLKGAVRQRERAEPEPCTCGPAGPVAASLAPVSATAGED |
| | | DDDVVGDV |
| 13 | hERFE (43- | GAGCCGCCGCCCGGGAACGAGCTGCCCCGGGGCCCCGGGGAGAGC |
| | 185) | CGCGCGGGGCCGGCCGCTCGTCCGCCGGAGCCCACCGCTGAGCGT |
| | C155/157S | GCACACAGCGTCGACCCCCGGGACGCCTGGATGCTCTTCGTCAGG |
| | cDNA | CAGAGTGACAAGGGTGTCAATGGCAAGAAGAGGAGCAGGGGCAAG |
| | | GCCAAGAAGCTGAAGTTCGGCTTGCCAGGGCCCCCTGGGCCTCCC |
| | | GGTCCCCAGGGCCCCCCAGGCCCCATCATCCCACCCGAGGCGCTG |
| | | CTGAAGGAGTTCCAGCTGCTGCTGAAAGGTGCGGTGCGGCAGCGG |
| | | GAGCGCGCGGAGCCCGAACCCAGTACGAGTGGCCCCGCCGGGCCG |
| | | GTCGCTGCGAGCCTCGCCCCGGTCTCGGCCACCGCCGGGGAGGAC |
| | | GACGACGACGTGGTGGGGGACGTGTGA |
| 14 | hERFE (43- | EPPPGNELPRGPGESRAGPAARPPEPTAERAHSVDPRDAWMLFVR |
| | 185) | QSDKGVNGKKRSRGKAKKLKFGLPGPPGPPGPQGPPGPIIPPEAL |
| | C155/157S | LKEFQLLLKGAVRQRERAEPEPSTSGPAGPVAASLAPVSATAGED |
| | amino acid | DDDVVGDV |

### Example 2: ERFE Fc-Fusion Protein Expression and Biophysical Property

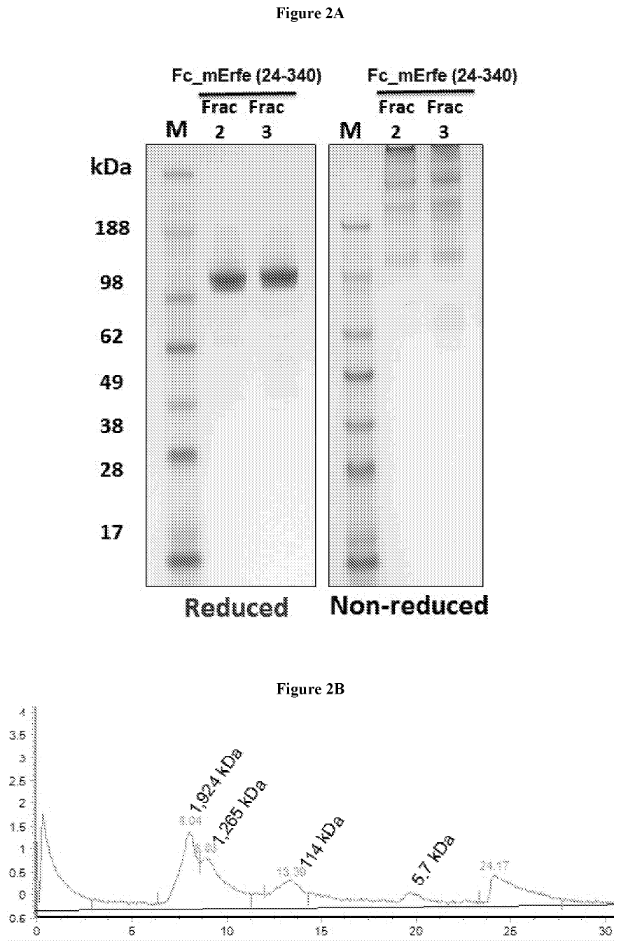

As a GPP repeat domain commonly drives protein homotrimerization, as well as coupling with the presence of a TNF-α like domain, ERFE protein alternatively exists as a homotrimer/multimeric protein in vivo. Consistently, recombinant full-length mERFE, Fc_mERFE (24-340), was found to predominantly form a mixture of higher order multimers. This was determined by SDS-PAGE under non-reducing conditions and size exclusion chromatography (SEC) (FIG. 2A and FIG. 2B).

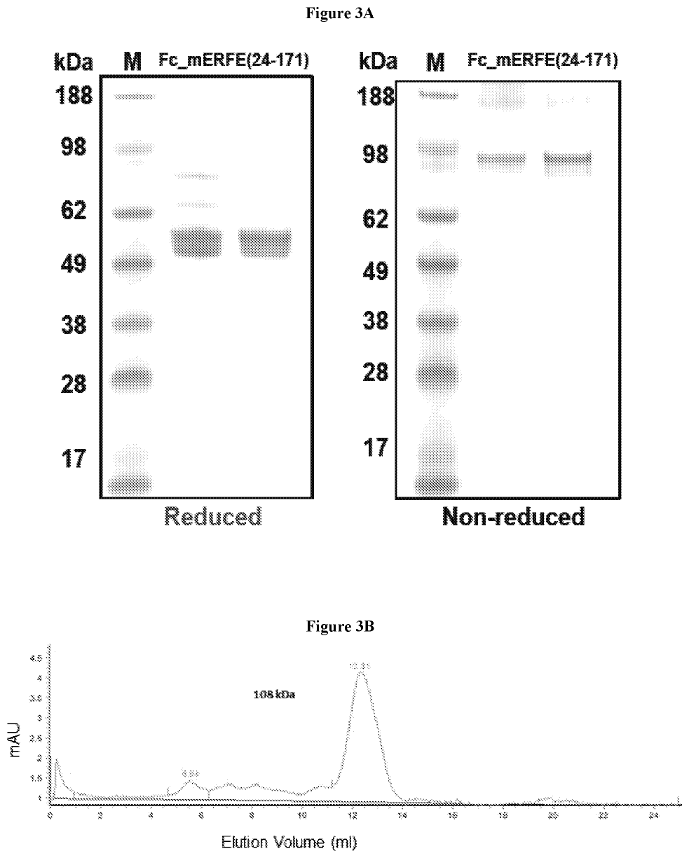

mERFE truncate, Fc_mERFE (24-171) was found to be active but ran as a doublet on SDS-PAGE under reducing conditions. Non-reducing gel and SEC revealed that Fc_mErfe (24-171) predominantly formed dimers (FIG. 3A and FIG. 3B).

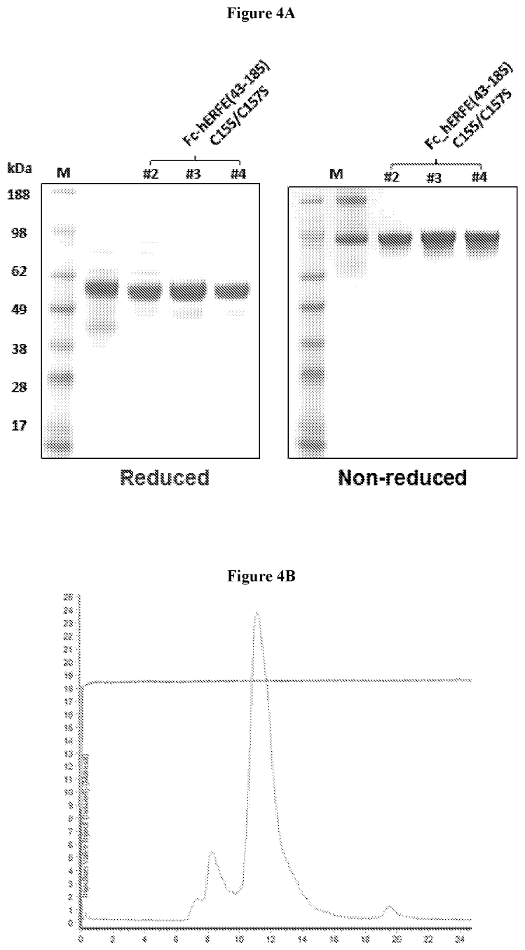

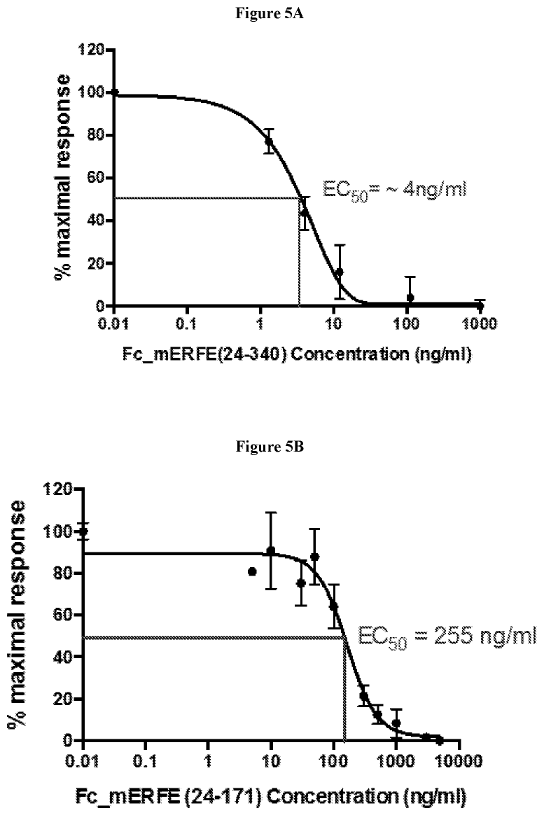

Fc_hERFE (43-185), the human homolog of Fc_mERFE (24-171), contains two free cysteines at amino acid position 155 and 157, respectively. The Fc_hERFE (43-185) fusion protein eliminates a protease cleavage site at residue 42. C155 and C157 were subsequently changed to serines, resulting in Fc_hERFE (43-185) C155/157S, which eliminates free cysteines and intermolecular disulfide bond formation. This construct was is transiently expressed at high levels and demonstrated advantageous biophysical properties (FIG. 4A and FIG. 4B).

### Example 3: In Vitro Functional Activity

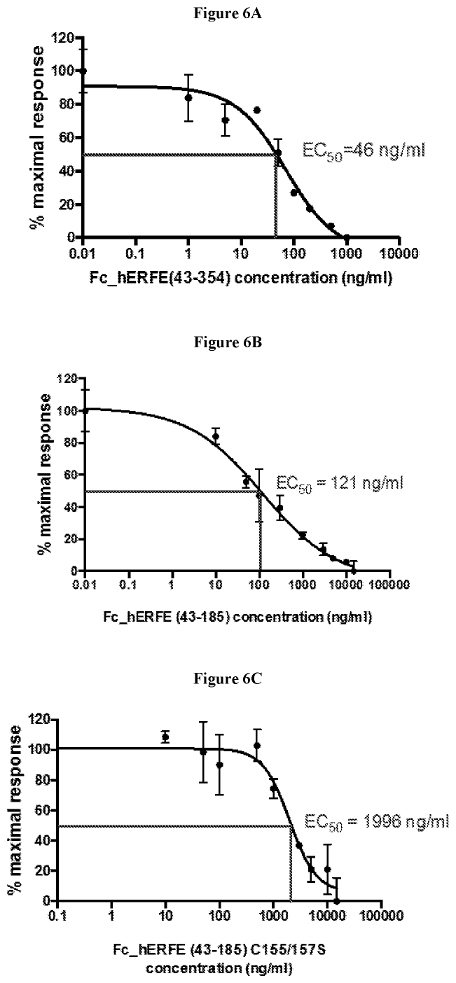

Hep3B human hepatoma cells were incubated with ERFE protein for 6 or 15 hrs. Cells were subsequently lysed and HAMP mRNA levels were determined by qRT-PCR relative to a reference control gene HPRT1. To determine the EC50 of ERFE protein, the relative HAMP expression was calculated for each concentration and then converted to a percentile response. Maximal HAMP expression is defined as the level of HAMP in samples receiving no ERFE treatment and zero HAMP represents the level of HAMP resulting from the maximum repression by ERFE. EC50 is the ERFE concentration that results in 50% repression of HAMP. Data are provided at FIG. 5A and FIG. 5B and FIG. 6A, FIG. 6B, and FIG. 6C for the various treatments.

### Example 4: In vivo activity

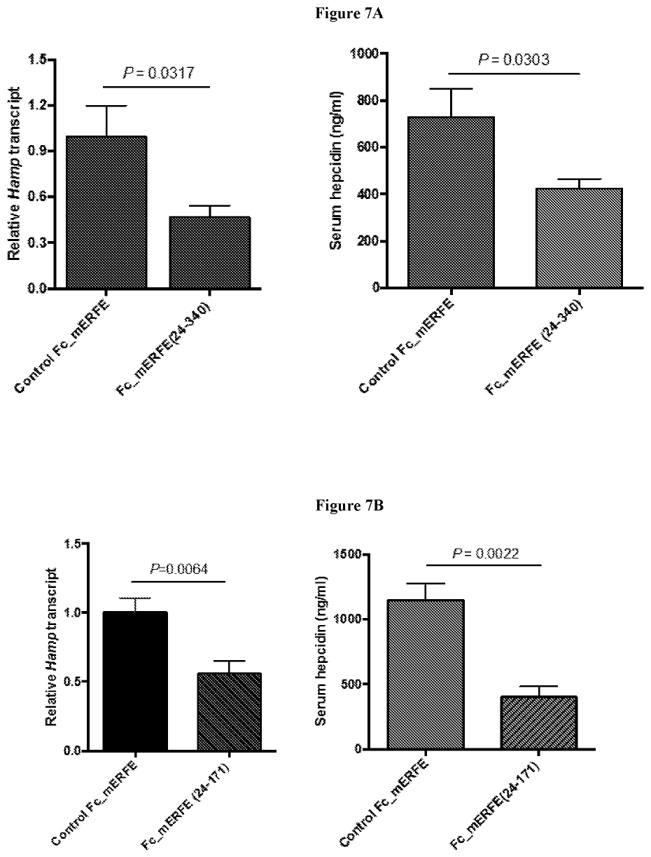

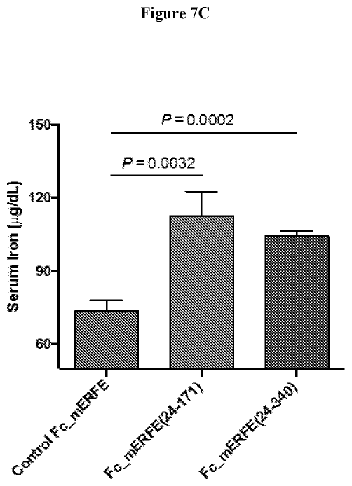

To determine if active mERFE proteins could reduce hepcidin levels in vivo, 6-week old C57BL/6J male mice were injected through intraperitoneal route with 10 mg/kg of mERFE Fc-fusion proteins, namely Fc_mERFE (24-340) and Fc_mERFE (24-171). An inactive mERFE Fc-fusion protein (Fc_mERFE (172-340) was used as negative control. At 6 hours post injection, serum and liver tissue were collected and analyzed for: a) hepcidin levels in the serum; b) Hamp transcript levels in the liver; and c) transferrin bound serum iron levels. Each experimental group consisted of 5-8 mice. The study demonstrated that both mERFE fusion polypeptides down regulated Hamp mRNA and serum hepcidin levels (FIG. 7A and FIG. 7B) at 6 hours, resulting in elevated serum iron levels (FIG. 7C).

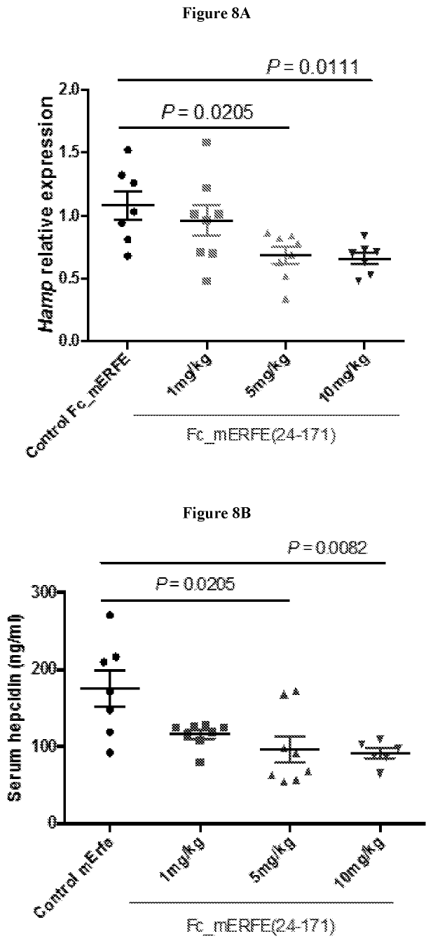

In addition, a dose-response of Fc_mERFE (24-171) was carried out using the same protocol. Three groups of mice (8 mice/group) were received 1 mg/kg, 5 mg/kg, and 10 mg/kg of Fc_mERFE (24-171), respectively; one group consisting of 8 mice received 1 mg/kg of inactive mERFE Fc-fusion protein as negative control. The study showed that at 5 mg/kg dosage Fc_mERFE (24-171) has exhibited significant suppressing effect on hepcidin (FIG. 8A and FIG. 8B).

### Example 5: In Vitro Functional Activity in the Presence of IL-6

Anemia of inflammation (AI) is a common feature of inflammatory disorders, including connective tissue diseases, infections, certain cancers, and chronic kidney disease. In inflammatory disorders and during infection, hepcidin synthesis is stimulated by proinflammatory cytokines, most prominently interleukin-6 (IL-6). Increased hepcidin causes hypoferremia and inadequate iron supply for erythropoiesis, resulting in iron-restricted anemia.

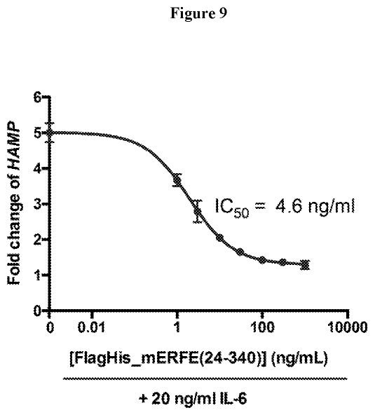

In this assay, Hep3B human hepatoma cells were incubated with (Flag)3-(His)6_mERFE (24-340) protein for 16 hrs in the presence of 20 ng/ml interleukin-6 (IL-6). Cells were subsequently lysed and HAMP mRNA levels were determined by qRT-PCR relative to a reference control gene HPRT1. Fold change of HAMP expression was calculated for cells treated with IL-6/ERFE in comparison to cells treated with buffer only. Data are provided at FIG. 9.

This example demonstrates that ERFE inhibits IL-6-mediated hepcidin induction in a dose-dependent manner in a Hep3B cellular HAMP suppression assay. The result implicates that by suppressing hepcidin and increasing iron availability, ERFE could be explored as a potential therapeutic intervention for anemia of inflammation.

What is claimed is:

1. A fusion polypeptide, comprising (a) an ERFE polypeptide having a sequence at least 85% identical to a fragment of SEQ ID NO: 2 or SEQ ID NO: 8, and (b) a heterologous polypeptide.

2. The fusion polypeptide of claim 1, wherein the ERFE polypeptide comprises at least a fragment of wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of SEQ ID NO: 2 or SEQ ID NO: 8.

3. The fusion polypeptide of claim 1, wherein the ERFE polypeptide consists of a fragment of wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of SEQ ID NO: 2 or SEQ ID NO: 8.

4. The fusion polypeptide of claim 1, wherein the ERFE polypeptide comprises about 140 to about 320 amino acids at least 85% identical to SEQ ID NO: 2 or SEQ ID NO: 8.

5. The fusion polypeptide of any one of claims 1 to 4, wherein the ERFE polypeptide has a sequence at least 85% identical to a polypeptide selected from the group consisting of SEQ ID NO: 4, SEQ ID NO: 6, SEQ ID NO: 10, SEQ ID NO: 12, and SEQ ID NO: 14.

6. The fusion polypeptide of any one of claims 1 to 5, wherein the ERFE polypeptide has a sequence at least 90% identical to a polypeptide selected from the group consisting of SEQ ID NO: 4, SEQ ID NO: 6, SEQ ID NO: 10, SEQ ID NO: 12, and SEQ ID NO: 14.

7. The fusion polypeptide of any one of claims 1 to 6, wherein the ERFE polypeptide has a sequence at least 95% identical to a polypeptide selected from the group consisting of SEQ ID NO: 4, SEQ ID NO: 6, SEQ ID NO: 10, SEQ ID NO: 12, and SEQ ID NO: 14.

8. The fusion polypeptide of any one of claims 1 to 7, wherein the ERFE polypeptide has a sequence 99% identical to a polypeptide selected from the group consisting of SEQ ID NO: 4, SEQ ID NO: 6, SEQ ID NO: 10, SEQ ID NO: 12, and SEQ ID NO: 14.

9. The fusion polypeptide of any one of claims 1 to 8, wherein the heterologous polypeptide is selected from the group consisting of calmodulin, polyglutamine, E-tag, FLAG, HA, His, Myc, S-tag, SBP-tag, Softag 1, Softag3, Strep-tag, TC-tag, V5, VSV, Xpress, Isopeptag, SpyTag, SnoopTag, BCCP, GST, GFP, Halo-tag, MBP, Nus-tag, Thioredoxin, albumin, an antibody, Fc domain, and combinations thereof.

10. The fusion polypeptide of any one of claims 1 to 9, wherein the heterologous polypeptide is an Fc domain.

11. The fusion polypeptide of claim 9, wherein the antibody comprises an anti-albumin antibody.

12. The fusion polypeptide of any one of claims 1 to 11, wherein the heterologous polypeptide is at the N-terminus of the ERFE polypeptide.

13. The fusion polypeptide of any one of claims 1 to 12, wherein the ERFE polypeptide has a sequence at least 85% identical to SEQ ID NO: 4 and the heterologous polypeptide comprises an Fc domain, wherein the heterologous polypeptide is fused or linked to the N-terminus of the ERFE polypeptide.

14. The fusion polypeptide of any one of claims 1 to 12, wherein the ERFE polypeptide has a sequence at least 85% identical to SEQ ID NO: 6 and the heterologous polypeptide comprises an Fc domain, wherein the heterologous polypeptide is fused or linked to the N-terminus of the ERFE polypeptide.

15. The fusion polypeptide of any one of claims 1 to 12, wherein the ERFE polypeptide has a sequence at least 85% identical to SEQ ID NO: 10 and the heterologous polypeptide comprises an Fc domain, wherein the heterologous polypeptide is fused or linked to the N-terminus of the ERFE polypeptide.

16. The fusion polypeptide of any one of claims 1 to 12, wherein the ERFE polypeptide has a sequence at least 85% identical to SEQ ID NO: 12 and the heterologous polypeptide comprises an Fc domain, wherein the heterologous polypeptide is fused or linked to the N-terminus of the ERFE polypeptide.

17. The fusion polypeptide of any one of claims 1 to 11, wherein the heterologous polypeptide is at the C-terminus of the ERFE polypeptide.

18. The fusion polypeptide of any one of claims 1 to 17, wherein the fusion polypeptide forms a homo-multimer.

19. The fusion polypeptide of claim 18, wherein the homo-multimer is a homodimer.

20. The fusion polypeptide of any one of claims 1 to 19, wherein the fusion polypeptide is glycosylated or phosphorylated.

21. A polynucleotide having a sequence that encodes the fusion polypeptide of any one of claims 1 to 20.

22. A composition comprising the fusion polypeptide of any one of claims 1 to 20 or the polynucleotide of claim 21, and an excipient.

23. The composition of claim 22, wherein the excipient comprises at least one of the group consisting of saline, maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

24. The composition of claim 22 or claim 23, further comprising an additional therapeutic agent.

25. The composition of claim 24, wherein the additional therapeutic agent is iron or erythropoietin.

26. The fusion polypeptide of any one of claims 1 to 20, the polynucleotide of claim 21, or the composition of any one of claims 22 to 25 for use as a medicament.

27. The fusion polypeptide of any one of claims 1 to 20, the polynucleotide of claim 21, or the composition of any one of claims 22 to 25 for use in treatment of a disease or disorder of iron metabolism.

28. The fusion polypeptide, the polynucleotide, or the composition of claim 27, wherein the disease or disorder of iron metabolism is selected from the group consisting of hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia, sideroblastic anemia, porphyria, porphyria cutanea tarda, African iron overload, hyperferritinemia, ceruloplasmin deficiency, atransferrinemia, congenital dyserythropoietic anemia, anemia of chronic disease, anemia of inflammation, anemia of infection, hypochromic microcytic anemia, iron-deficiency anemia, iron-restricted anemia, iron-refractory iron deficiency anemia, anemia of chronic kidney disease, erythropoietin resistance, iron deficiency of obesity, and other anemias.

29. The fusion polypeptide, the polynucleotide, or the composition of claim 27, wherein the disease or disorder of iron metabolism is anemia of inflammation, anemia of chronic diseases, anemia of chronic kidney disease, and iron-restricted anemia.

30. The fusion polypeptide, the polynucleotide, or the composition of claim 27, wherein the disease or disorder of iron metabolism is iron-restricted anemia.

31. The fusion polypeptide, the polynucleotide, or the composition of claim 27, wherein the disease or disorder of iron metabolism is anemia of chronic disease.

32. The fusion polypeptide, the polynucleotide, or the composition of claim 27, wherein the disease or disorder of iron metabolism is anemia of inflammation.

33. The fusion polypeptide, the polynucleotide, or the composition of claim 27, wherein the disease or disorder of iron metabolism is anemia of chronic kidney disease.

34. A pharmaceutical composition, comprising an ERFE fusion polypeptide and an excipient.

35. The pharmaceutical composition of claim 34, wherein the fusion protein comprises (a) an ERFE polypeptide having a sequence at least 85% identical to a fragment of SEQ ID NO: 2 or SEQ ID NO: 8 and (b) a heterologous polypeptide.

36. The pharmaceutical composition of claim 35, wherein the ERFE polypeptide comprises at least a fragment of wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of SEQ ID NO: 2 or SEQ ID NO: 8.

37. The pharmaceutical composition of claim 35, wherein the ERFE polypeptide consists of a fragment of wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of SEQ ID NO: 2 or SEQ ID NO: 8.

38. The pharmaceutical composition of claim 35, wherein the ERFE polypeptide comprises about 140 to about 320 amino acids at least 85% identical to SEQ ID NO: 2 or SEQ ID NO: 8.

39. The pharmaceutical composition of any one of claims 35 to 38, wherein the ERFE polypeptide has a sequence at least 85% identical to a polypeptide selected from the group consisting of SEQ ID NO: 4, SEQ ID NO: 6, SEQ ID NO: 10, SEQ ID NO: 12, and SEQ ID NO: 14.

40. The pharmaceutical composition of any one of claims 35 to 38, wherein the ERFE polypeptide has a sequence at least 90% identical to a polypeptide selected from the group consisting of SEQ ID NO: 4, SEQ ID NO: 6, SEQ ID NO: 10, SEQ ID NO: 12, and SEQ ID NO: 14.

41. The pharmaceutical composition of any one of claims 35 to 38, wherein the ERFE polypeptide has a sequence at least 95% identical to a polypeptide selected from the group consisting of SEQ ID NO: 4, SEQ ID NO: 6, SEQ ID NO: 10, SEQ ID NO: 12, and SEQ ID NO: 14.

42. The pharmaceutical composition of any one of claims 35 to 38, wherein the ERFE polypeptide has a sequence 99% identical to a polypeptide selected from the group consisting of SEQ ID NO: 4, SEQ ID NO: 6, SEQ ID NO: 10, SEQ ID NO: 12, and SEQ ID NO: 14.

43. The pharmaceutical composition of any one of claims 35 to 42, wherein the heterologous polypeptide is selected from the group consisting of calmodulin, polyglutamine, E-tag, FLAG, HA, His, Myc, S-tag, SBP-tag, Softag 1, Softag3, Strep-tag, TC-tag, V5, VSV, Xpress, Isopeptag, SpyTag, SnoopTag, BCCP, GST, GFP, Halo-tag, MBP, Nus-tag, Thioredoxin, albumin, an antibody, Fc domain, and combinations thereof.

44. The pharmaceutical composition of any one of claims 35 to 43, wherein the heterologous polypeptide is an Fc domain.

45. The pharmaceutical composition of claim 43, wherein the antibody comprises an anti-albumin antibody.

46. The pharmaceutical composition of any one of claims 35 to 45, wherein the heterologous polypeptide is at the N-terminus of the ERFE polypeptide.

47. The pharmaceutical composition of any one of claims 35 to 45, wherein the heterologous polypeptide is at the C-terminus of the ERFE polypeptide.

48. The pharmaceutical composition of any one of claims 34 to 47, wherein the ERFE fusion polypeptide forms a homo-multimer.

49. The pharmaceutical composition of claim 48, wherein the homo-multimer is a homodimer.

50. The pharmaceutical composition of any one of claims 35 to 49, wherein the ERFE polypeptide comprises a modification selected from the group consisting of a glycosylation and a phosphorylation.

51. The pharmaceutical composition of any one of claims 35 to 50, wherein the excipient comprises at least one of the group consisting of saline, maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

52. The pharmaceutical composition of claim 35 to claim 51, comprising an additional therapeutic agent.

53. The pharmaceutical composition of claim 52, wherein the additional therapeutic agent comprises iron or erythropoietin.

54. A method of treating a disease or disorder of iron metabolism in an individual in need thereof, comprising administering to the individual a therapeutically-effective amount of an ERFE fusion polypeptide.

55. The method of claim 54, wherein the ERFE fusion polypeptide comprises (a) an ERFE polypeptide comprising an ERFE polypeptide having a sequence at least 85% identical to a fragment of SEQ ID NO: 2 or SEQ ID NO: 8 and (b) a heterologous polypeptide.

56. The method of claim 55, wherein the ERFE polypeptide comprises at least a fragment of wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of SEQ ID NO: 2 or SEQ ID NO: 8.

57. The method of claim 55, wherein the ERFE polypeptide consists of a fragment of wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of SEQ ID NO: 2 or SEQ ID NO: 8.

58. The method of claim 55, wherein the ERFE polypeptide comprises about 140 to about 320 amino acids at least 85% identical to SEQ ID NO: 2 or SEQ ID NO: 8.

59. The method of any one of claims 55 to 58, wherein the ERFE polypeptide has a sequence at least 85% identical to a polypeptide selected from the group consisting of SEQ ID NO: 4, SEQ ID NO: 6, SEQ ID NO: 10, SEQ ID NO: 12, and SEQ ID NO: 14.

60. The method of any one of claims 55 to 58, wherein the ERFE polypeptide has a sequence at least 90% identical to a polypeptide selected from the group consisting of SEQ ID NO: 4, SEQ ID NO: 6, SEQ ID NO: 10, SEQ ID NO: 12, and SEQ ID NO: 14.

61. The method of any one of claims 55 to 58, wherein the ERFE polypeptide has a sequence at least 95% identical to a polypeptide selected from the group consisting of SEQ ID NO: 4, SEQ ID NO: 6, SEQ ID NO: 10, SEQ ID NO: 12, and SEQ ID NO: 14.

62. The method of any one of claims 55 to 58, wherein the ERFE polypeptide has a sequence 99% identical to a polypeptide selected from the group consisting of SEQ ID NO: 4, SEQ ID NO: 6, SEQ ID NO: 10, SEQ ID NO: 12, and SEQ ID NO: 14.

63. The method of any one of claims 55 to 62, wherein the heterologous polypeptide is selected from the group consisting of calmodulin, polyglutamine, E-tag, FLAG, HA, His, Myc, S-tag, SBP-tag, Softag 1, Softag3, Strep-tag, TC-tag, V5, VSV, Xpress, Isopeptag, SpyTag, SnoopTag, BCCP, GST, GFP, Halo-tag, MBP, Nus-tag, Thioredoxin, albumin, an antibody, Fc domain, and combinations thereof.

64. The method of any one of claims 55 to 63, wherein the heterologous polypeptide is an Fc domain.

65. The method of claim 63, wherein the antibody comprises an anti-albumin antibody.

66. The method of claim 63, wherein the antibody targets an ERFE polypeptide to a specific cell or tissue.

67. The method of any one of claims 55 to 66, wherein the heterologous polypeptide is at the N-terminus of the ERFE polypeptide.

68. The method of any one of claims 55 to 66, wherein the heterologous polypeptide is at the C-terminus of the ERFE polypeptide.

69. The method of any one of claims 54 to 68, wherein the ERFE fusion polypeptide forms a homo-multimer.

70. The method of claim 69, wherein the homo-multimer is a homodimer.

71. The method of any one of claims 54 to 70, wherein the ERFE fusion polypeptide comprises a modification selected from the group consisting of a glycosylation and a phosphorylation.

72. The method of claim any one of claims 54 to 71, wherein the ERFE fusion polypeptide comprises a composition comprising an excipient.

73. The method of claim 72, wherein the excipient comprises at least one of the group consisting of maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

74. The method of any one of claims 54 to 73, further comprising administering to the individual at least one an additional therapeutic agent.

75. The method of claim 74, wherein the additional therapeutic agent is iron or erythropoietin.

76. The method of any one of claims 54 to 75, wherein the disease or disorder of iron metabolism is selected from the group consisting of hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia, sideroblastic anemia, porphyria, porphyria cutanea tarda, African iron overload, hyperferritinemia, ceruloplasmin deficiency, and atransferrinemia.

77. The method of any one of claims 54 to 75, wherein the disease or disorder of iron metabolism is selected from the group consisting of congenital dyserythropoietic anemia, anemia of chronic disease, anemia of inflammation, anemia of infection, hypochromic microcytic anemia, iron-deficiency anemia, iron-restricted anemia, iron-refractory iron deficiency anemia, anemia of chronic kidney disease, erythropoietin resistance, iron deficiency of obesity, and other anemias.

78. The method of any one of claims 54 to 75, wherein the disease or disorder of iron metabolism is selected from the group consisting of anemia of inflammation, anemia of chronic disease, anemia of chronic kidney disease, and iron-restricted anemia.

79. The method of any one of claims 54 to 75, wherein the disease or disorder of iron metabolism is iron-restricted anemia.

80. The method of any one of claims 54 to 75, wherein the disease or disorder of iron metabolism is anemia of chronic disease.

81. The method of any one of claims 54 to 75, wherein the disease or disorder of iron metabolism is anemia of inflammation.

82. The method of any one of claims 54 to 75, wherein the disease or disorder of iron metabolism is anemia of chronic kidney disease.

83. The method of any one of claims 54 to 82, wherein the method reduces at least one symptom of a disease or disorder of iron metabolism.

84. The method of claim 83, wherein the symptom is selected from the group consisting of chronic fatigue, joint pain, abdominal pain, liver disease (cirrhosis, liver cancer), diabetes mellitus, irregular heart rhythm, heart attack or heart failure, skin color changes (bronze, ashen-gray green), loss of menstrual period, loss of interest in sex, osteoarthritis, osteoporosis, hair loss, enlarged liver or spleen, impotence, infertility, hypogonadism, hypothyroidism, hypopituitarism, depression, adrenal function problems, early onset neurodegenerative disease, elevated blood sugar, elevated liver enzymes, elevated iron (serum iron, serum ferritin), weakness, pale skin, shortness of breath, dizziness, dietary cravings, tingling or crawling feeling in the legs, tongue swelling or soreness, cold hands and feet, fast or irregular heartbeat, brittle nails, and headache.

85. A kit comprising an ERFE fusion polypeptide and at least one buffer or excipient.

86. The kit of claim 85, wherein the ERFE fusion polypeptide comprises (a) an ERFE polypeptide having a sequence at least 85% identical to a fragment of SEQ ID NO: 2 or SEQ ID NO: 8 and (b) a heterologous polypeptide.

87. The kit of claim 86, wherein the ERFE polypeptide comprises at least a fragment of wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of SEQ ID NO: 2 or SEQ ID NO: 8.

88. The kit of claim 86, wherein the ERFE polypeptide consists of a fragment of wildtype ERFE having at least about 10, 20, 30, 40, 50, 60, 70, 80, 90, 100, 110, 120, 130, 140, 150, 160, 170, 180, 190, 200, 210, 220, 230, 240, 250, 260, 270, 280, 290, 300, 310, 320, 330, 340, or more amino acids of SEQ ID NO: 2 or SEQ ID NO: 8.

89. The kit of claim 86, wherein the ERFE polypeptide comprises about 140 to about 320 amino acids at least 85% identical to SEQ ID NO: 2 or SEQ ID NO: 8.

90. The kit of any one of claims 86 to 89, wherein the ERFE polypeptide has a sequence at least 85% identical to a polypeptide selected from the group consisting of SEQ ID NO: 4, SEQ ID NO: 6, SEQ ID NO: 10, SEQ ID NO: 12, and SEQ ID NO: 14.

91. The kit of any one of claims 86 to 89, wherein the ERFE polypeptide has a sequence at least 90% identical to a polypeptide selected from the group consisting of SEQ ID NO: 4, SEQ ID NO: 6, SEQ ID NO: 10, SEQ ID NO: 12, and SEQ ID NO: 14.

92. The kit of any one of claims 86 to 89, wherein the ERFE polypeptide has a sequence at least 95% identical to a polypeptide selected from the group consisting of SEQ ID NO: 4, SEQ ID NO: 6, SEQ ID NO: 10, SEQ ID NO: 12, and SEQ ID NO: 14.

93. The kit of any one of claims 86 to 89, wherein the ERFE polypeptide has a sequence 99% identical to a polypeptide selected from the group consisting of SEQ ID NO: 4, SEQ ID NO: 6, SEQ ID NO: 10, SEQ ID NO: 12, and SEQ ID NO: 14.

94. The kit of any one of claims 86 to 93, wherein the heterologous polypeptide is selected from the group consisting of calmodulin, polyglutamine, E-tag, FLAG, HA, His, Myc, S-tag, SBP-tag, Softag 1, Softag3, Strep-tag, TC-tag, V5, VSV, Xpress, Isopeptag, SpyTag, SnoopTag, BCCP, GST, GFP, Halo-tag, MBP, Nus-tag, Thioredoxin, albumin, an antibody, Fc domain, and combinations thereof.

95. The kit of any one of claims 86 to 94, wherein the heterologous polypeptide is an Fc domain.

96. The kit of claim 94, wherein the antibody comprises an anti-albumin antibody.

97. The kit of claim 94, wherein the antibody targets the ERFE polypeptide to a specific cell or tissue.

98. The kit of any one of claims 86 to 97, wherein the heterologous polypeptide is at the N-terminus of the ERFE polypeptide.

99. The kit of any one of claims 86 to 97, wherein the heterologous polypeptide is at the C-terminus of the ERFE polypeptide.

100. The kit of any one of claims 85 to 99, wherein the ERFE fusion polypeptide forms a homo-multimer.

101. The kit of claim 100, wherein the homo-multimer is a homodimer.

102. The kit of any one of claims 86 to 101, wherein the ERFE polypeptide comprises a modification selected from the group consisting of a glycosylation and a phosphorylation.

103. The kit of claims 85 to 102, wherein the excipient comprises at least one of the group consisting of maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and surfactant polyoxyethylene-sorbitan monooleate.

104. The kit of any one of claims 85 to 103, comprising at least one an additional therapeutic agent.

105. The kit of claim 104, wherein the additional therapeutic agent comprises iron or erythropoietin.

106. The kit of any one of claims 85 to 105, comprising written instructions for treating a disease or disorder of iron metabolism selected from the group consisting of hemochromatosis, HFE mutation hemochromatosis, ferroportin mutation hemochromatosis, transferrin receptor 2 mutation hemochromatosis, hemojuvelin mutation hemochromatosis, hepcidin mutation hemochromatosis, juvenile hemochromatosis, neonatal hemochromatosis, hepcidin deficiency, transfusional iron overload, thalassemia, thalassemia intermedia, alpha thalassemia, sideroblastic anemia, porphyria, porphyria cutanea tarda, African iron overload, hyperferritinemia, ceruloplasmin deficiency, atransferrinemia, congenital dyserythropoietic anemia, anemia of chronic disease, anemia of inflammation, anemia of infection, hypochromic microcytic anemia, iron-deficiency anemia, iron-restricted anemia, iron-refractory iron deficiency anemia, anemia of chronic kidney disease, erythropoietin resistance, iron deficiency of obesity, and other anemias.

107. The kit of any one of claims 85 to 105, comprising written instructions for treating a disease or disorder of iron metabolism selected from the group consisting of iron-restricted anemia, anemia of chronic disease, anemia of inflammation, and anemia of chronic kidney disease.

108. The kit of any one of claims 85 to 105, comprising written instructions for treating iron-restricted anemia.

109. The kit of any one of claims 85 to 105, comprising written instructions for treating anemia of chronic disease.

110. The kit of any one of claims 85 to 105, comprising written instructions for treating anemia of inflammation.

111. The kit of any one of claims 85 to 105, comprising written instructions for treating anemia of chronic kidney disease.

112. A fusion polypeptide, comprising (a) an ERFE polypeptide having a sequence of SEQ ID NO: 4, and (b) a Fc domain, wherein the Fc domain is fused to the N-terminus of the ERFE polypeptide.

113. A fusion polypeptide, comprising (a) an ERFE polypeptide having a sequence of SEQ ID NO: 6, and (b) a Fc domain, wherein the Fc domain is fused to the N-terminus of the ERFE polypeptide.

114. A fusion polypeptide, comprising (a) an ERFE polypeptide having a sequence of SEQ ID NO: 10, and (b) a Fc domain, wherein the Fc domain is fused to the N-terminus of the ERFE polypeptide.

115. A fusion polypeptide, comprising (a) an ERFE polypeptide having a sequence of SEQ ID NO: 12, and (b) a Fc domain, wherein the Fc domain is fused to the N-terminus of the ERFE polypeptide.

116. A fusion polypeptide, comprising (a) an ERFE polypeptide having a sequence of SEQ ID NO: 14, and (b) a Fc domain, wherein the Fc domain is fused to the N-terminus of the ERFE polypeptide.

117. The fusion polypeptide of any one of claims 112 to 116, wherein the Fc domain has a sequence of SEQ ID NO: 40.

118. A fusion polypeptide, comprising (a) an ERFE polypeptide having a sequence of SEQ ID NO: 4, and (b) a (Flag)3-(His)6 domain, wherein the (Flag)3-(His)6 domain comprises three Flag domains and six histidine residues and wherein the (Flag)3-(His)6 domain is fused to the N-terminus of the ERFE polypeptide.

119. The fusion polypeptide of claim 118, wherein the Flag domain has a sequence of SEQ ID NO: 18.

120. The fusion polypeptide of claim 118 or claim 119, wherein the (His)6 domain has a sequence of SEQ ID NO: 20.


SEQUENCE LISTING (40 sequences)

SEQ ID NO: 1 - DNA, 1023 bp, Mus musculus
atggcctcgacccgccgccccgtcggagctcgcacgctgctcgcctgcgccagcctactcgccgccatgggcctcggtgtccctgagtccgcggagcccgtggggactcatgcacgcccgcagccgcccggggccgagctgcccgccccgccagccaacagcccgccggaacccaccattgcgcatgcacacagtgtggatccccgggatgcttggatgctgttcgtcaagcagagtgacaaggggatcaacagtaagaggaggagcaaagccaggaggctgaagcttggcctgccaggacccccagggccaccaggtcctcagggccccccaggcccctttatcccatctgaggttctgctgaaggagttccagctgttgctgaaaggcgcagtacggcagcgagagagccatctggagcactgcaccagggatctcactacaccagcctcgggtagcccttcccgtgtcccagccgcccaggagcttgatagccaggacccaggggcattgttagctctgctggctgcgaccttggcccagggcccgcgggcaccacgtgtggaggccgcattccactgtcgcttgcgccgggatgtgcaggtggatcggcgtgcgttgcacgagcttgggatctactacctgcccgaagttgagggagccttccaccggggcccaggcttgaatctgaccagcggccagtacaccgcacctgtggctggcttctatgcgcttgctgccactctgcacgtggcactcaccgagcagccaagaaagggaccaacacgaccccgggatcgtctgcgcctgctgatctgcatccagtctctctgccagcacaatgcctccctggagactgtgatggggctggagaacagcagcgagctcttcaccatctcagtaaatggtgtcctctatctacaggcaggacactacacttctgtcttcttggacaatgccagcggctcctccctcacggtacgcagtggctctcacttcagtgctatcctcctgggcctgtga

SEQ ID NO: 2 - protein, 340 aa, Mus musculus
MASTRRPVGARTLLACASLLAAMGLGVPESAEPVGTHARPQPPGAELPAPPANSPPEPTIAHAHSVDPRDAWMLFVKQSDKGINSKRRSKARRLKLGLPGPPGPPGPQGPPGPFIPSEVLLKEFQLLLKGAVRQRESHLEHCTRDLTTPASGSPSRVPAAQELDSQDPGALLALLAATLAQGPRAPRVEAAFHCRLRRDVQVDRRALHELGIYYLPEVEGAFHRGPGLNLTSGQYTAPVAGFYALAATLHVALTEQPRKGPTRPRDRLRLLICIQSLCQHNASLETVMGLENSSELFTISVNGVLYLQAGHYTSVFLDNASGSSLTVRSGSHFSAILLGL

SEQ ID NO: 3 - DNA, 957 bp, Mus musculus
atgggcctcggtgtccctgagtccgcggagcccgtggggactcatgcacgcccgcagccgcccggggccgagctgcccgccccgccagccaacagcccgccggaacccaccattgcgcatgcacacagtgtggacccccgggatgcttggatgctgttcgtcaagcagagtgacaaggggatcaacagtaagaggaggagcaaagccaggaggctgaagcttggcctgccaggacccccagggccaccaggtcctcagggccccccaggcccctttatcccatctgaggttctgctgaaggagttccagctgttgctgaaaggcgcagtacggcagcgagagagccatctggagcactgcaccagggatctcactacaccagcctcgggtagcccttcccgtgtcccagccgcccaggagcttgatagccaggacccaggggcattgttagctctgctggctgcgaccttggcccagggcccgcgggcaccacgtgtggaggccgcattccactgtcgcttgcgccgggatgtgcaggtggatcggcgtgcgttgcacgagcttgggatctactacctgcccgaagttgagggagccttccaccggggcccaggcttgaatctgaccagcggccagtacaccgcacctgtggctggcttctatgcgcttgctgccactctgcacgtggcactcaccgagcagccaagaaagggaccaacacgaccccgggatcgtctgcgcctgctgatctgcatccagtctctctgccagcacaatgcctccctggagactgtgatggggctggagaacagcagcgagctcttcaccatctcagtaaatggtgtcctctatctacaggcaggacactacacttctgtcttcttggacaatgccagcggctcctccctcacggtacgcagtggctctcacttcagtgctatcctcctgggcctgtga

SEQ ID NO: 4 - protein, 318 aa, Mus musculus
MGLGVPESAEPVGTHARPQPPGAELPAPPANSPPEPTIAHAHSVDPRDAWMLFVKQSDKGINSKRRSKARRLKLGLPGPPGPPGPQGPPGPFIPSEVLLKEFQLLLKGAVRQRESHLEHCTRDLTTPASGSPSRVPAAQELDSQDPGALLALLAATLAQGPRAPRVEAAFHCRLRRDVQVDRRALHELGIYYLPEVEGAFHRGPGLNLTSGQYTAPVAGFYALAATLHVALTEQPRKGPTRPRDRLRLLICIQSLCQHNASLETVMGLENSSELFTISVNGVLYLQAGHYTSVFLDNASGSSLTVRSGSHFSAILLGL

SEQ ID NO: 5 - DNA, 450 bp, Mus musculus
atgggcctcggtgtccctgagtccgcggagcccgtggggactcatgcacgcccgcagccgcccggggccgagctgcccgccccgccagccaacagcccgccggaacccaccattgcgcatgcacacagtgtggacccccgggatgcttggatgctgttcgtcaagcagagtgacaaggggatcaacagtaagaggaggagcaaagccaggaggctgaagcttggcctgccaggacccccagggccaccaggtcctcagggccccccaggcccctttatcccatctgaggttctgctgaaggagttccagctgttgctgaaaggcgcagtacggcagcgagagagccatctggagcactgcaccagggatctcactacaccagcctcgggtagcccttcccgtgtcccagccgcccaggagcttgatagccaggacccaggggcattgtga

SEQ ID NO: 6 - protein, 149 aa, Mus musculus
MGLGVPESAEPVGTHARPQPPGAELPAPPANSPPEPTIAHAHSVDPRDAWMLFVKQSDKGINSKRRSKARRLKLGLPGPPGPPGPQGPPGPFIPSEVLLKEFQLLLKGAVRQRESHLEHCTRDLTTPASGSPSRVPAAQELDSQDPGAL

SEQ ID NO: 7 - DNA, 1065 bp, Homo sapiens
atggccccggcccgccgccccgccggagcccgcctgctgctcgtctacgcgggcctgctggccgccgccgccgcgggcctggggtccccggagcctggggcgccctcgaggagccgcgcccgcagggagccgccgcccgggaacgagctgccccggggccccggggagagccgcgcggggccggccgctcgtccgccggagcccaccgctgagcgtgcacacagcgtcgacccccgggacgcctggatgctcttcgtcaggcagagtgacaagggtgtcaatggcaagaagaggagcaggggcaaggccaagaagctgaagttcggcttgccagggccccctgggcctcccggtccccagggccccccaggccccatcatcccacccgaggcgctgctgaaggagttccagctgctgctgaaaggtgcggtgcggcagcgggagcgcgcggagcccgaaccctgtacgtgtggccccgccgggccggtcgctgcgagcctcgccccggtctcggccaccgccggggaggacgacgacgacgtggtgggggacgtgctggcactgctggccgcgcccctggccccggggccgcgggcgccgcgcgtggaggccgctttcctctgccgcctgcgccgggacgcgttggtggagcggcgcgcgctgcacgagcttggcgtctactacctgcccgacgccgagggtgccttccgccgcggcccgggcctgaacttgaccagcggccagtacagggcgcccgtggctggcttctacgctctcgccgccacgctgcacgtggcgctcggggagccgccgaggagggggccgccgcgcccccgggaccacctgcgcctgctcatctgcatccagtcccggtgccagcgcaacgcctccctggaggccatcatgggcctggagagcagcagtgagctcttcaccatctctgtgaatggcgtcctgtacctgcagatggggcagtggacctccgtgttcttggacaacgccagcggctgctccctcacagtgcgcagtggctcccacttcagtgctgtcctcctgggcgtgtga

SEQ ID NO: 8 - protein, 354 aa, Homo sapiens
MAPARRPAGARLLLVYAGLLAAAAAGLGSPEPGAPSRSRARREPPPGNELPRGPGESRAGPAARPPEPTAERAHSVDPRDAWMLFVRQSDKGVNGKKRSRGKAKKLKFGLPGPPGPPGPQGPPGPIIPPEALLKEFQLLLKGAVRQRERAEPEPCTCGPAGPVAASLAPVSATAGEDDDDVVGDVLALLAAPLAPGPRAPRVEAAFLCRLRRDALVERRALHELGVYYLPDAEGAFRRGPGLNLTSGQYRAPVAGFYALAATLHVALGEPPRRGPPRPRDHLRLLICIQSRCQRNASLEAIMGLESSSELFTISVNGVLYLQMGQWTSVFLDNASGCSLTVRSGSHFSAVLLGV

SEQ ID NO: 9 - DNA, 939 bp, Homo sapiens
gagccgccgcccgggaacgagctgccccggggccccggggagagccgcgcggggccggccgctcgtccgccggagcccaccgctgagcgtgcacacagcgtcgacccccgggacgcctggatgctcttcgtcaggcagagtgacaagggtgtcaatggcaagaagaggagcaggggcaaggccaagaagctgaagttcggcttgccagggccccctgggcctcccggtccccagggccccccaggccccatcatcccacccgaggcgctgctgaaggagttccagctgctgctgaaaggtgcggtgcggcagcgggagcgcgcggagcccgaaccctgtacgtgtggccccgccgggccggtcgctgcgagcctcgccccggtctcggccaccgccggggaggacgacgacgacgtggtgggggacgtgctggcactgctggccgcgcccctggccccggggccgcgggcgccgcgcgtggaggccgctttcctctgccgcctgcgccgggacgcgttggtggagcggcgcgcgctgcacgagcttggcgtctactacctgcccgacgccgagggtgccttccgccgcggcccgggcctgaacttgaccagcggccagtacagggcgcccgtggctggcttctacgctctcgccgccacgctgcacgtggcgctcggggagccgccgaggagggggccgccgcgcccccgggaccacctgcgcctgctcatctgcatccagtcccggtgccagcgcaacgcctccctggaggccatcatgggcctggagagcagcagtgagctcttcaccatctctgtgaatggcgtcctgtacctgcagatggggcagtggacctccgtgttcttggacaacgccagcggctgctccctcacagtgcgcagtggctcccacttcagtgctgtcctcctgggcgtgtga

SEQ ID NO: 10 - protein, 312 aa, Homo sapiens
EPPPGNELPRGPGESRAGPAARPPEPTAERAHSVDPRDAWMLFVRQSDKGVNGKKRSRGKAKKLKFGLPGPPGPPGPQGPPGPIIPPEALLKEFQLLLKGAVRQRERAEPEPCTCGPAGPVAASLAPVSATAGEDDDDVVGDVLALLAAPLAPGPRAPRVEAAFLCRLRRDALVERRALHELGVYYLPDAEGAFRRGPGLNLTSGQYRAPVAGFYALAATLHVALGEPPRRGPPRPRDHLRLLICIQSRCQRNASLEAIMGLESSSELFTISVNGVLYLQMGQWTSVFLDNASGCSLTVRSGSHFSAVLLGV

SEQ ID NO: 11 - DNA, 432 bp, Homo sapiens
gagccgccgcccgggaacgagctgccccggggccccggggagagccgcgcggggccggccgctcgtccgccggagcccaccgctgagcgtgcacacagcgtcgacccccgggacgcctggatgctcttcgtcaggcagagtgacaagggtgtcaatggcaagaagaggagcaggggcaaggccaagaagctgaagttcggcttgccagggccccctgggcctcccggtccccagggccccccaggccccatcatcccacccgaggcgctgctgaaggagttccagctgctgctgaaaggtgcggtgcggcagcgggagcgcgcggagcccgaaccctgtacgtgtggccccgccgggccggtcgctgcgagcctcgccccggtctcggccaccgccggggaggacgacgacgacgtggtgggggacgtgtga

SEQ ID NO: 12 - protein, 143 aa, Homo sapiens
EPPPGNELPRGPGESRAGPAARPPEPTAERAHSVDPRDAWMLFVRQSDKGVNGKKRSRGKAKKLKFGLPGPPGPPGPQGPPGPIIPPEALLKEFQLLLKGAVRQRERAEPEPCTCGPAGPVAASLAPVSATAGEDDDDVVGDV

SEQ ID NO: 13 - DNA, 432 bp, Homo sapiens
gagccgccgcccgggaacgagctgccccggggccccggggagagccgcgcggggccggccgctcgtccgccggagcccaccgctgagcgtgcacacagcgtcgacccccgggacgcctggatgctcttcgtcaggcagagtgacaagggtgtcaatggcaagaagaggagcaggggcaaggccaagaagctgaagttcggcttgccagggccccctgggcctcccggtccccagggccccccaggccccatcatcccacccgaggcgctgctgaaggagttccagctgctgctgaaaggtgcggtgcggcagcgggagcgcgcggagcccgaacccagtacgagtggccccgccgggccggtcgctgcgagcctcgccccggtctcggccaccgccggggaggacgacgacgacgtggtgggggacgtgtga

SEQ ID NO: 14 - protein, 143 aa, Homo sapiens
EPPPGNELPRGPGESRAGPAARPPEPTAERAHSVDPRDAWMLFVRQSDKGVNGKKRSRGKAKKLKFGLPGPPGPPGPQGPPGPIIPPEALLKEFQLLLKGAVRQRERAEPEPSTSGPAGPVAASLAPVSATAGEDDDDVVGDV

SEQ ID NO: 15 - protein, 149 aa, Unknown [sequence omitted]

SEQ ID NO: 16 - protein, 6 aa, Artificial Sequence
  Description of Artificial Sequence Synthetic peptide
EEEEEE

SEQ ID NO: 17 - protein, 13 aa, Artificial Sequence
  Description of Artificial Sequence Synthetic peptide
GAPVPYPDPLEPR

SEQ ID NO: 18 - protein, 8 aa, Artificial Sequence
  Description of Artificial Sequence Synthetic peptide
DYKDDDDK

SEQ ID NO: 19 - protein, 9 aa, Artificial Sequence
  Description of Artificial Sequence Synthetic peptide
YPYDVPDYA

SEQ ID NO: 20 - protein, 6 aa, Artificial Sequence
  Description of Artificial Sequence Synthetic 6xHis tag
HHHHHH

SEQ ID NO: 21 - protein, 10 aa, Artificial Sequence
  Description of Artificial Sequence Synthetic peptide
EQKLISEEDL

SEQ ID NO: 22 - protein, 18 aa, Artificial Sequence
  Description of Artificial Sequence Synthetic peptide
TKENPRSNQEESYDDNES

SEQ ID NO: 23 - protein, 15 aa, Artificial Sequence
  Description of Artificial Sequence Synthetic peptide
KETAAAKFERQHMDS

SEQ ID NO: 24 - protein, 38 aa, Artificial Sequence
  Description of Artificial Sequence Synthetic polypeptide
MDEKTTGWRGGHVVEGLAGELEQLRARLEHHPQGQREP

SEQ ID NO: 25 - protein, 13 aa, Artificial Sequence
  Description of Artificial Sequence Synthetic peptide
SLAELLNAGLGGS

SEQ ID NO: 26 - protein, 8 aa, Artificial Sequence
  Description of Artificial Sequence Synthetic peptide
TQDPSRVG

SEQ ID NO: 27 - protein, 8 aa, Artificial Sequence
  Description of Artificial Sequence Synthetic peptide
WSHPQFEK

SEQ ID NO: 28 - protein, 6 aa, Artificial Sequence
  Description of Artificial Sequence Synthetic peptide
CCPGCC

SEQ ID NO: 29 - protein, 14 aa, Artificial Sequence
  Description of Artificial Sequence Synthetic peptide
GKPIPNPLLGLDST

SEQ ID NO: 30 - protein, 11 aa, Artificial Sequence
  Description of Artificial Sequence Synthetic peptide
YTDIEMNRLGK

SEQ ID NO: 31 - protein, 8 aa, Artificial Sequence
  Description of Artificial Sequence Synthetic peptide
DLYDDDDK

SEQ ID NO: 32 - protein, 16 aa, Artificial Sequence
  Description of Artificial Sequence Synthetic peptide
TDKDMTITFTNKKDAE

SEQ ID NO: 33 - protein, 13 aa, Artificial Sequence
  Description of Artificial Sequence Synthetic peptide
AHIVMVDAYKPTK

SEQ ID NO: 34 - protein, 12 aa, Artificial Sequence
  Description of Artificial Sequence Synthetic peptide
KLGDIEFIKVNK

SEQ ID NO: 35 - protein, 83 aa, Unknown
  Description of Unknown BCCP polypeptide
AAAEISGHIVRSPMVGTFYRTPSPDAKAFIEVGQKVNVGDTLCIVEAMKMMNQIEADKSGTVKAILVESGQPVEFDEPLVVIE

SEQ ID NO: 36 - protein, 232 aa, Unknown
  Description of Unknown GST polypeptide
MSPILGYWKIKGLVQPTRLLLEYLEEKYEEHLYERDEGDKWRNKKFELGLEFPNLPYYIDGDVKLTQSMAIIRYIADKHNMLGGCPKERAEISMLEGAVLDIRYGVSRIAYSKDFETLKVDFLSKLPEMLKMFEDRLCHKTYLNGDHVTHPDFMLYDALDVVLYMDPMCLDAFPKLVCFKKRIEAIPQIDKYLKSSKYIAWPLQGWQATFGGGDHPPKSDLVPRGSPGIHRD

SEQ ID NO: 37 - protein, 239 aa, Unknown
  Description of Unknown GFP polypeptide
MVSKGEELFTGVVPILVELDGDVNGHKFSVSGEGEGDATYGKLTLKFICTTGKLPVPWPTLVTTLTYGVQCFSRYPDHMKQHDFFKSAMPEGYVQERTIFFKDDGNYKTRAEVKFEGDTLVNRIELKGIDFKEDGNILGHKLEYNYNSHNVYIMADKQKNGIKVNFKIRHNIEDGSVQLADHYQQNTPIGDGPVLLPDNHYLSTQSALSKDPNEKRDHMVLLEFVTAAGITLGMDELYK

SEQ ID NO: 38 - protein, 387 aa, Unknown
  Description of Unknown MBP polypeptide
MKIEEGKLVIWINGDKGYNGLAEVGKKFEKDTGIKVTVEHPDKLEEKFPQVAATGDGPDIIFWAHDRFGGYAQSGLLAEITPDKAFQDKLYPFTWDAVRYNGKLIAYPIAVEALSLIYNKDLLPNPPKTWEEIPALDKELKAKGKSALMFNLQEPYFTWPLIAADGGYAFKYENGKYDIKDVGVDNAGAKAGLTFLVDLIKNKHMNADTDYSIAEAAFNKGETAMTINGPWAWSNIDTSKVNYGVTVLPTFKGQPSKPFVGVLSAGINAASPNKELAKEFLENYLLTDEGLEAVNKDKPLGAVALKSYEEELAKDPRIAATMENAQKGEIMPNIPQMSAFWYAVRTAVINAASGRQTVDEALKDAQTNSSSNNNNNNNNNNLGIEGR

SEQ ID NO: 39 - protein, 609 aa, Unknown [sequence omitted]

SEQ ID NO: 40 - protein, 232 aa, Unknown
  Description of Unknown Fc polypeptide
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK